(19) United States
(12) Patent Application Publication
(10) Pub. No.: US 2025/0041668 A1
(43) Pub. Date: Feb. 6, 2025

(54) EXERCISE MENU MANAGEMENT DEVICE, EXERCISE MANAGEMENT METHOD, AND COMPUTER PROGRAM

(71) Applicant: ASAHI INTECC CO., LTD., Seto-shi (JP)

(72) Inventors: Yasuhiro KANOKO, Seto-shi (JP); Kasuhiko KAWASHITA, Tokyo (JP); Tatsuya WATANABE, Tokyo (JP)

(73) Assignee: ASAHI INTECC CO., LTD., Seto-shi (JP)

(21) Appl. No.: 18/920,408

(22) Filed: Oct. 18, 2024

Related U.S. Application Data
(63) Continuation of application No. PCT/JP2023/014170, filed on Apr. 6, 2023.

(30) Foreign Application Priority Data
Apr. 19, 2022 (JP) 2022-069097

(51) Int. Cl. A63B 24/00; A63B 71/06; G16H 20/30; G16H 40/67

(52) U.S. Cl. CPC A63B 24/0075; A63B 71/0619; G16H 20/30; G16H 40/67; A63B 2071/0675

(57) ABSTRACT

An exercise menu contributing to improvement of a walking function is created and is provided to a user. An exercise menu management device includes a memory storing body state information, walking importance degree information, and exercise information. A processor selects one or more of the predetermined parts on the basis of the body state information and the walking importance degree information, creates the exercise menu by selecting a predetermined exercise related to the selected one or more predetermined parts, and outputs the exercise menu that has been created.

This application is a bypass continuation of International Application No. PCT/JP2023/014170 filed Apr. 6, 2023, which is based upon and claims the benefit of priority from Japanese Patent Application No. 2022-069097 filed Apr. 19, 2022, the entire contents of the prior applications being incorporated herein by reference.

### TECHNICAL FIELD

The present disclosure relates to an exercise menu management device, an exercise management method, and a computer program.

### BACKGROUND ART

A training system (Patent Literature 1) and a training menu presentation system (Patent Literature 2) for the purpose of slimming and muscle enhancement are known.

### CITATION LIST

### Patent Literature

[Patent Literature 1]Japanese Patent No. 6623822
[Patent Literature 2]Japanese Patent Application Laid-Open No. 2018-166885

### SUMMARY

### Technical Problem

In an aging society, healthy longevity is more desired than slimming and muscle enhancement. It is known that the walking function and healthy longevity have a close relationship. However, there is no known technology of creating an exercise menu useful for improving the walking function and providing a user with the exercise menu.

The present disclosure has been made in view of the above problem and has an object to provide an exercise menu management device, an exercise management method, and a computer program that are capable of creating an exercise menu contributing to improvement of a walking function and providing a user with the exercise menu.

### Solution to Problem

In order to solve the above problem, an exercise menu creation device according to an aspect of the present disclosure is a device that provides a user with an exercise menu related to improvement of a walking function, the device including: a body state information storage part that stores body state information being information of a plurality of predetermined parts related to the walking function among parts of a body of the user; a walking importance degree information storage part that stores walking importance degree information indicating a degree of importance related to the walking function for each of the plurality of predetermined parts; an exercise information storage part that stores exercise information indicating a relationship between a plurality of exercises contributing to improvement of the walking function and the plurality of predetermined parts; an exercise menu creation part that selects one or more of the plurality of predetermined parts on the basis of the body state information and the walking importance degree information, and creates the exercise menu by selecting a predetermined exercise related to the predetermined parts selected from a plurality of the exercises; and an output part that outputs the exercise menu that has been created.

### Advantageous Effects

According to the present disclosure, it is possible to provide a user with an exercise menu related to improvement of a walking function.

### BRIEF DESCRIPTION OF DRAWINGS

FIG. 1 is an overall schematic view of an exercise menu management system.

FIG. 2 is a system configuration diagram of the exercise menu management system.

FIG. 3 is an example of user basic information.

FIG. 4 is an example of body state information.

FIG. 5 is an example of walking importance degree information.

FIG. 6 is an example of exercise information.

FIG. 7 is an example of exercise moving image management information.

FIG. 8 is an example of an exercise execution record.

FIG. 9 is an example of a walking record.

FIG. 10 is an example of walking evaluation standard.

FIG. 11 is a flowchart of exercise menu management processing.

FIG. 12 is a flowchart of body state evaluation processing in FIG. 11.

FIG. 13 is a flowchart illustrating exercise menu creation processing in FIG. 11.

FIG. 14 is a flowchart illustrating exercise moving image distribution processing in FIG. 11.

FIG. 15 is a flowchart of exercise activity data acquisition processing in FIG. 11.

FIG. 16 is a flowchart of reminder transmission processing in FIG. 11.

FIG. 17 is an example of an exercise menu management screen displayed in a user device.

FIG. 18 is a flowchart of event management processing according to Example 2.

FIG. 19 is a flowchart of processing of evaluating walking data.

FIG. 20 is a flowchart indicating exercise moving image distribution processing according to Example 3.

FIG. 21 is a flowchart indicating processing of evaluating body state information according to Example 4.

FIG. 22 is a flowchart of processing of reproducing an exercise moving image in the user device according to Example 5.

FIG. 23 is a flowchart indicating processing of an exercise device according to Example 6.

FIG. 24 is an overall schematic view of the exercise menu management system according to Example 7.

### DESCRIPTION OF EMBODIMENTS

Hereinafter, embodiments of the present disclosure will be described with reference to the drawings. An exercise menu management system according to the present embodiment creates an exercise menu useful for improvement of a walking function of a user on the basis of a body state of the user and provides the user with the exercise menu. Here, improvement of the walking function in this description includes not only improvement or enhancement of the walking function but also maintaining of the walking function and preventing the walking function from decreasing.

The user executes an exercise indicated in the exercise menu provided from the exercise menu management system. When executing the exercise, the user can use a user device and an exercise device. A result of execution of the exercise by the user is recorded in the exercise menu management system.

### Example 1

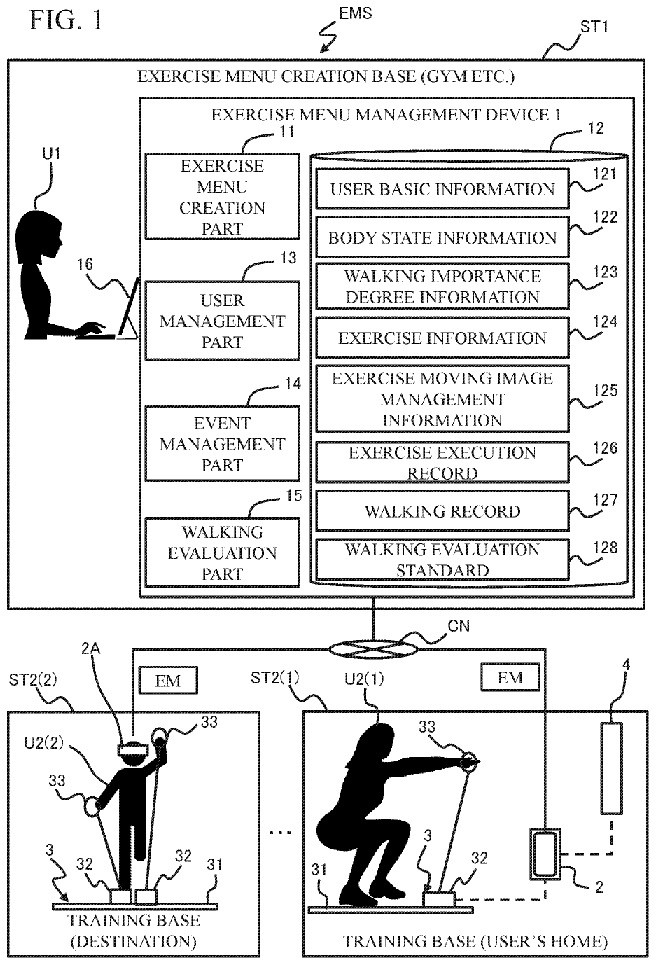

Example 1 will be described with reference to FIGS. 1 to 17. FIG. 1 is an overall schematic view of an exercise menu management system EMS. The exercise menu management system EMS includes, for example, an exercise menu management device 1, at least one user device 2, and an exercise device 3. The exercise menu management device 1 and each user device 2 are connected to each other such that bidirectional communication can be performed via a communication network CN such as the Internet, for example.

An exercise menu management device 1 is, for example, provided in an exercise menu creation base ST1 such as a sport gym. The exercise menu management device 1 is operated by an exercise manager U1 such as a trainer of the exercise. The exercise manager U1 may be a trainer who coaches the exercise of a user U2, and may be an operator who operates the exercise menu management device 1 according to an instruction by the trainer. The exercise manager U1 may be a doctor, a nurse, a physical therapist, an occupational therapist, or the like.

The user device 2 and the exercise device 3 are provided in a training base ST2 where the user U2 executes the exercise. The user U2 is a user of an exercise menu management service provided by an exercise menu management system EMS. The training base ST2 is not a sport gym which the exercise manager U1 belongs to, and is, for example, a home of the user U2, a destination of the user, or the like. The destination of the user U2 is, for example, a home of a friend or acquaintance, a workplace, a park, a hotel, a commercial facility, or a hospital.

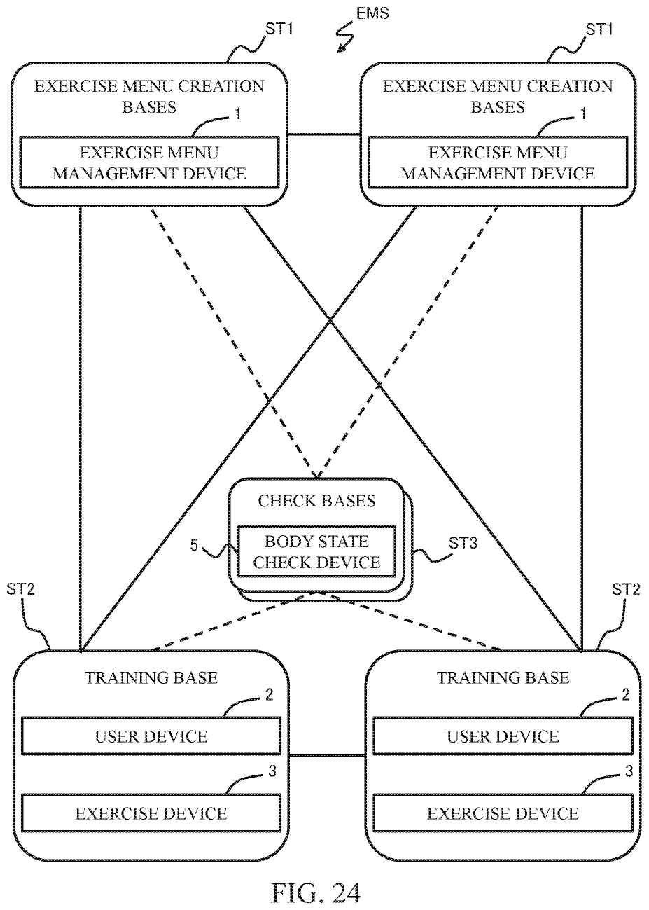

As described later, a check base ST3 (see FIG. 24) for checking the body state information of the user U2 may be provided in the exercise menu management system EMS.

The user U2 can go outside while carrying the user device 2 and the exercise device 3 and execute the exercise in a place other than his/her home. The user U2 also can go outside while carrying only the user device 2 and execute the exercise in a place other than his/her home. Moreover, the user U2 can go outside without carrying the user device 2 and the exercise device 3 and execute the exercise using the user device 2 and the exercise device 3 placed in a place other than the home.

First, the user device 2 and the exercise device 3 will be described, and then, the exercise menu management device 1 will be described.

The user device 2 is an information processing terminal used by a user, such as a laptop type personal computer, a tablet type personal computer, a desktop type personal computer, a tablet type information terminal, a mobile phone (including a so-called smart phone), and a wearable information terminal. The user device 2 may be configured by one device or may be configured by interlocking of a plurality of devices. For example, the user device 2 may be configured by interlocking of a wristwatch type wearable terminal and a smartphone.

In the training base ST2(1) illustrated on the right side of FIG. 1, the user device 2 is connected to a television device 4 via a wire or wirelessly. An exercise moving image received by the user device 2 from the exercise menu management device 1 via the communication network CN is transferred to the television device 4 and displayed.

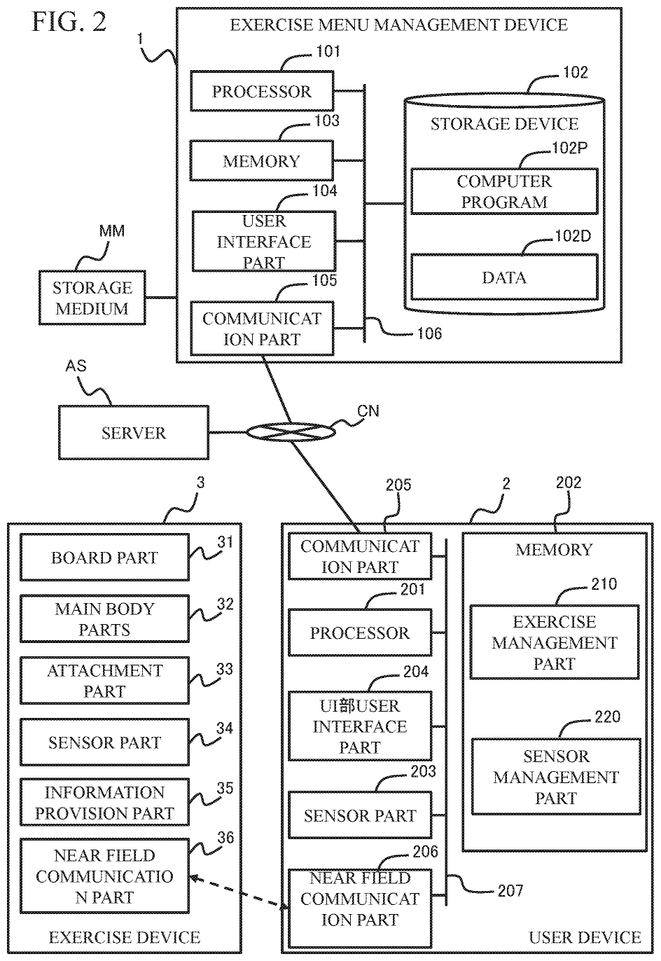

When the exercise device 3 has a communication function, the user device 2 can be connected also to the exercise device 3 so as to be able to perform communication via a wire or wirelessly. As described later, the user device 2 can acquire data from a sensor part 34 (see FIG. 2) provided in the exercise device 3. The user device 2 can transmit the data detected by the sensor part 203 of the user device 2 and the data received from the exercise device 3 to the exercise menu management device 1 via the communication network CN.

In the training base ST2(2) illustrated on the left side of FIG. 1, the user device 2A is configured as a goggle type device that provides a visual sense of the user with a world different from the real world. The goggle type device 2A provides the user with a world different from the real world called the virtual reality (VR), the augmented reality (AR), the mixed reality (MR), the extended reality (ER), or the like. The user can do exercise in a virtual world, and can check a numerical value such as a body temperature or a heartrate in a normal visual field while doing exercise in the real world. The user can do exercise while watching a motion of an exercise trainer that appears virtually as a three-dimensional object. The user also can do exercise while the exercise moving image received from the exercise menu management device 1 is displayed in the real world or the virtual world.

The exercise device 3 will be described. The exercise device 3 includes, for example, a board part 31, a plurality of main body parts 32 provided on the board part 31, and an attachment part 33 provided in each of the main body parts 32 so as to be expandable and attached to the body of the user.

When the exercise device 3 is a device for one person, two main body parts 32 are provided in the board part 31 such that the main body parts 32 is attachable to and detachable from the board part 31. When the exercise device 3 is configured as a device for two people, four main body parts 32 are provided in the board part 31. Accordingly, although there is no limitation in the number of main body parts 32 included in the exercise device 3, in the description below, the exercise device 3 of this example includes two body parts 32 in consideration of convenience at the time of carrying and storage.

In this example, in consideration of convenience at the time of carrying and storage of the exercise device 3, each main body part 32 is provided in the board part 31 such that each main body part 32 is attachable to and detachable from the board part 31. Each main body part 32 can be detachably attached to the board part 31 by fixation means such as a magnet, an adhesive, a screw, a fastener, a fitting structure of a recess and a projection, a clamp mechanism. However, any one or a plurality of the main body parts 32 may be fixed to the board part 31 such that the main body part 32 is undetachable. In this case, work of attaching the main body part 32 to the board part 31 can be eliminated, so that convenience of the user U2 is improved.

Each main body part 32 applies a force of pulling the attachment part 33 separated from the main body part 32 to the main body part 32. For example, each main body part 32 incorporates a mechanism part (not illustrated) such as flat spiral spring, and a proximal end side of the attachment part 33 is connected to the mechanism part. At least a part of the attachment part 33 is wound in the inside of the main body part 32. When the attachment part 33 is pulled out by the user U2, a force (here, referred to as a restoring force) of returning the pulled-out attachment part 33 to an original position acts. The mechanism part may use power other than the flat spiral spring. A motor or a gear may be used as the mechanism part. When the mechanism part uses electricity as a drive source, a battery cell may be incorporated, a power device using a commercially available power via a tap may be provided, and a device using power supplied from the outside utilizing electric waves, an induced electromotive force, or light may be provided.

The attachment part 33 is attached to the body of the user U2 in such a manner that the user U2 grips the attachment part 33, for example. The attachment part 33 may be attached not only to the hand of the user U2 but also to the arm, the ankle, the leg, the thigh, the waist, or the like of the user U2. The user U2 may grip two attachment parts 33 with the right and left hands, and the user U2 may grip the two attachment parts 33 with one of the right and left hands.

The exercise menu management device 1 will be described. The exercise menu management device 1 is configured as a computer system as described later, and function parts 11 to 15 described below are implemented by hardware resources and software resources of the computer system.

The exercise menu management device 1 includes, for example, an exercise menu creation part 11, a storage part 12, a user management part 13, an event management part 14, a walking evaluation part 15, a user interface device for manager 16.

The exercise menu creation part 11 has a function of selecting one or more predetermined parts among a plurality of predetermined parts on the basis of body state information and walking importance degree information, selecting a predetermined exercise related to the selected predetermined parts among a plurality of exercises, and creating an exercise menu EM.

The exercise menu creation part 11 has a function of managing the body state information, a function of managing the walking importance degree information, a function of managing the exercise information, the function of managing the exercise moving image, a function of managing the exercise execution record, and a function of managing the walking record. These body state information management function, walking importance degree information management function, exercise information management function, exercise moving image management function, exercise execution record management function, and walking record management function may be illustrated in the outside of the exercise menu creation part 11, but are not illustrated in FIG. 1.

The storage part 12 has a function of storing various information used for operation of the exercise menu management service. The storage part 12 stores, for example, user basic information 121, body state information 122, walking importance degree information 123, exercise information 124, exercise moving image management information 125, exercise execution record 126, walking record 127, and a walking evaluation standard 128.

Each piece of information 121 to 128 may be referred to as a storage part that stores the information 121 to 128. For example, the user basic information 121 may be referred to as a user basic information storage part 121, the body state information 122 may be referred to as a body state information storage part 122, the walking importance degree information 123 may be referred to as a walking importance degree information storage part 123, the exercise information 124 may be referred to as an exercise information storage part 124, the exercise moving image management information 125 may be referred to as an exercise moving image management information storage part 125, the exercise execution record 126 may be referred to as an exercise execution record storage part 126, the walking record 127 may be referred to as a walking record storage part 127, and the walking evaluation standard 128 may be referred to as a walking evaluation standard storage part 128. Content of each piece of information 121 to 128 will be described later.

The user management part 13 has a function of managing each user U2 who uses the exercise menu management service. As described later, the user management part 13 can transmit a reminder related to execution of an exercise to the user device 2 for the user U2 on the basis of the exercise execution record 126 or transmit a reminder related to an event related to walking to the user device 2.

When information on health of the user U2, for example, sleeping time, content and amount of meal, or vital data can be acquired, the user management part 13 may give an advice to the user U2 about meal, sleeping, or the like on the basis of the health-related information.

The event management part 14 manages information on an event related to walking. The event related to walking is, for example, an event contributing to improvement of a walking function such as hiking, mountain walking, stroll, or the like. The event management part 14 manages date and time, place, the number of participants, a walking state of each participant, or the like. The event management part 14 can create a walking menu related to the user U2 who participates in the event and transmit the walking menu to the user device 2. The walking menu at the time of event may be created separately from the exercise menu created by the exercise menu creation part 11 and transmitted to the user device 2 at a different timing. The walking menu at the time of the event may be transmitted to the user device 2 together with the exercise menu.

The walking evaluation part 15 reads the walking record 127 and the walking evaluation standard 128 from the storage part 12 and evaluates the walking of the user U2. The exercise menu creation part 11 may select an exercise on the basis of the walking evaluation result and the body state information 122 and create the exercise menu. That is, the exercise menu management device 1 can create and provide the exercise menu appropriate for the user U2 by evaluating not only the exercise executed by the user U2 but also the walking state in daily life of the user U2 such as commuting to a workplace, commuting to a school, traveling, leisure, or the like.

The user interface device for manager 16 is a device operated by the exercise manager U1. The user interface device for manager 16 includes, for example, an information input device (not illustrated) that inputs information to the exercise menu management device 1, and an information output device (not illustrated) that outputs information from the exercise menu management device 1 and provides the exercise manager U1 with the information.

The information input device is, for example, a pointing device such as a keyboard or a mouse, a touch panel, a microphone, a sound recognition device, or a combination of these. The information output device is, for example, a monitor display, a printer, a speaker, a sound synthesis device, or a combination of these. The user interface device 16 may be a goggle type device that provides the exercise manager U1 with a visual world different from the real world such as the user device 2A. The exercise manager U1 can refer to the body state or the exercise execution record of the user U2 in the virtual space and create the exercise menu.

In this example, a case where the exercise manager U1 performs various determination such as evaluation of the body state information 122, evaluation of the exercise execution record 126, selection of the exercise, or evaluation of the walking record. However, the present disclosure is not limited thereto and artificial intelligence may be used for determination. Otherwise, the exercise manager U1 may perform final determination with reference to determination by artificial intelligence. For example, by causing a neural network to learn large number of pieces of training data that has been determined to be correct or not in advance by a human, determination can be made in a manner similar to a human when any data is input. Accordingly, by inputting the body state information 122 and the exercise execution record 126 of many users U2 and evaluation by a trainer, or the like to the neural network and causing the neural network to learn the data, determination on the exercise can be obtained. Deep learning may be used instead of the neural network.

FIG. 2 is a system configuration diagram of the exercise menu management system. The exercise menu management device 1 includes, for example, a processor 101, a storage device 102, a memory 103, a user interface (UI in the drawing) part 104, and a communication part 105, which are connected via communication means 106 such as a bus.

The processor 101 is not limited to a central processing unit and may include a processor that performs processing specialized for graphic operation or the like. The storage device 102 is, for example, an auxiliary storage device such as a flash memory or a hard disk drive, and stores a computer program 102P and data 102D. The memory 103 is a read only memory (ROM) and a random access memory (RAM). The memory 103 also provides the processor 101 with a work region. The processor 101 reads a computer program 102P and data 102D from the storage device 102 and stores them in the memory 103. By the processor 101 executing the computer program 102P using the data 102D, the functions 11 to 15 described in FIG. 1 are implemented. As similar to this, other functional parts described later are also implemented by the processor 101 using the computer program 102P and the data 102D.

The user interface part 104 is a circuit that transmits and receives information to and from the user interface device 16 used by the exercise manager U1. The communication part 105 is a circuit that performs bidirectional communication with the user device 2 via the communication network CN. The communication part 105 can perform communication directly or indirectly with a near field communication part 36 of the exercise device 3 via a communication part 205 and a near field communication part 206 of the user device 2 described later. When the exercise device 3 includes a communication part (not illustrated) that is connected to the communication network CN, the exercise device 3 can perform communication directly with the exercise menu management device 1 not via the user device 2.

The user device 2 is a device such as a smartphone or tablet terminal as described above and is used by the user device U2. The user device 2 may be a personal item of the user U2 or the exercise menu management service may lend the user device 2 to the user U2. A plurality of users U2 may use one user device 2. The user device 2 used by the user U2 may be changed to the other user device 2 regularly or irregularly.

The user device 2 includes, for example, a processor 201, a memory 202, a sensor part 203, a user interface part 204, a communication part 205, and a near field communication part 206, which are connected via communication means 207 such as a bus.

The processor 201 may include a processor that performs processing specialized for graphic operation or the like. The memory 202 here includes a read only memory (ROM), a random access memory (RAM), and a storage (auxiliary storage device). The memory 202 stores a computer program and data that implement the exercise management part 210 and the sensor management part 220.

The exercise management part 210 has a function for the user to use the exercise menu management service. The exercise management part 210 acquires the exercise menu from the exercise menu management device 1 according to an instruction of the user, acquires, from the exercise menu management device 1, a moving image (exercise moving image) being an example of the exercise selected by the user, and reproduces the moving image. The exercise management part 210 also transmits sensing data acquired from the sensor management part 220 to the exercise menu management device 1.

The sensor management part 220 acquires and stores the data detected by the sensor part 203 and causes the data to be transmitted to the exercise menu management device 1 via the exercise management part 210. The sensor management part 220 can transmit the data acquired from the sensor part 34 of the exercise device 3 to the exercise management part 210.

The sensor part 203 is, for example, an image sensor, an acceleration sensor, a position information sensor, a temperature sensor, a microphone, an optical sensor, a temperature sensor, a pressure sensor, or a pulse sensor. The sensor part 203 may be a combination of a plurality of sensors. The sensor part 203 may be an incorporated sensor incorporated in the user device 2 or an external sensor connected to the user device 2. The user device 2 may be a combination of an incorporated sensor and an external sensor. For example, a camera installed indoors may be used as an external sensor and moving image data captured by the camera may be used as sensor data.

The user interface part 204 is a device that enables information exchange between the user U2 and the user device 2. The user interface part 204 is configured like a touch panel capable of performing simultaneously input and output of information. The present disclosure is not limited to this, and a sound recognition device, a sound synthesis device, or the like may be used as the user interface part 204.

The communication part 205 is a circuit for communication with the exercise menu management device 1 via the communication network CN. The near field communication part 206 is a circuit for communication with the near field communication part 36 of the exercise device 3. The near field communication part 206 performs data communication with the exercise device 3 wirelessly, optically, or using a sound wave.

The exercise device 3 is a device used when the user U2 does exercise. The exercise device 3 includes, for example, a board part 31, a main body part 32, an attachment part 33, a sensor part 34, an information provision part 35, and a near field communication part 36.

The board part 31 is a support part for detachably attaching one or more main body parts 32. As described above, each main body part 32 generates a force (restoring force) of returning the attachment part 33 pulled by the user U2. The attachment part 33 is attached to the body of the user U2. The user U2 performs training of pulling the attachment part 33 from the main body part 32 in a state of standing on the board part 31. With the exercise device 3 described above, the user U2 becomes highly conscious of a reactive force of a sole transmitted from the board part 31 by a restoring force of the attachment part 33 of returning, so that an exercise that is more effective for improvement of the walking function can be provided.

The sensor part 34 is, for example, a pressure sensor, a vibration sensor, a temperature sensor, or the like provided in the board part 31, and detects a part of the body state information of the user U2 before the exercise, during the exercise, and after the exercise. That is, the sensor part 34 may detect not only the body state of the user U2 during the exercise but also the body state before the start of the exercise and the body state after the end of the exercise. The data detected in the sensor part 34 is transmitted from the near field communication part 36 to the near field communication part 206 of the user device 2. The sensor part 34 may be an image sensor.

The information provision part 35 provides the user with the information related to the exercise. The information provision part 35 provides the user U2 with the information related to the exercise by, for example, any one of an image, sound, or light such as a monitor display, a projector, a or speaker, or a combination of an image, sound, or light. The information related to the exercise is, for example, a moving image being an example (exercise moving image), an evaluation result of the state of the exercise, information for supporting the exercise. The evaluation result of the state of the exercise is information as to whether loads of both feet of the user U2 are equal to each other and whether any one of the loads of the feet is larger than the other. The information for supporting the exercise is information such as yells, the sound of applause, a blink of a light, or the like.

When the exercise device 3 includes a communication part (not illustrated) that is connected to the communication network CN, the exercise device 3 can perform communication directly with the exercise menu management device 1 not via the user device 2. Alternatively, a configuration may be adopted in which the exercise device 3 and the user device 2 are integrated and functions of the user device 2 are provided in the exercise device 3.

The server AS is a server computer that distributes a computer program or data to the exercise menu management device 1 and/or the user device 2. The user U2 can download the computer program for implementing the exercise management part 210 by accessing the server AS by using the user device 2. The exercise menu management device 1 can acquire, from the server AS, various information related to the exercise such as a weather forecast of a place where the user device 2 exists, news, information on nutrition of food, or the like and use the information for creating the exercise menu. The exercise device 3 may receive the computer program or data from the server AS.

The storage medium MM is a non-transitory storage medium storing a computer program such as a flash memory, a hard disk, an optical disk, or a magnetic tape. The storage medium MM and the storage device 102 can transmit and receive a computer program and data to and from each other. For example, at least a part of the computer program 102P or the data 102D can be transferred from the storage medium MM to the storage device 102 and stored in the storage device 102. On the contrary, at least a part of the computer program 102P or the data 102D can be transferred from the storage device 102 to the storage medium MM and stored in the storage medium MM. Here, at least a part of the computer program 102P or the data 102D refers to the entire computer program 102P, a part of the computer program 102P, the entire data 102D, a part of the data 102D, and a combination of these.

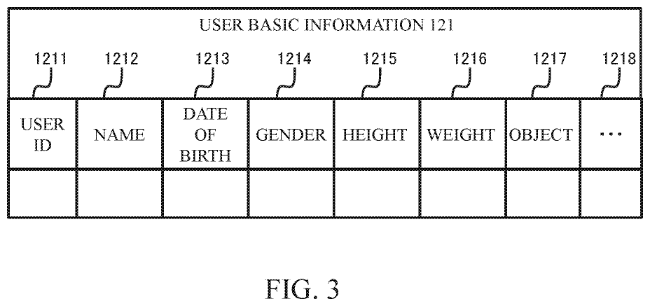

Referring to FIGS. 3 to 10, a configuration example of data used in the exercise menu management system EMS will be described. All items described in each piece of data described below are not essential and some of them may be omitted. Each data may include items other than the items explicitly described. Existence of items not explicitly described is represented in an item, “Others”.

FIG. 3 is an example of the user basic information 121. The user basic information 121 manages basic information on each user U2 who uses the exercise menu management service. The user basic information 121 can includes, for example, a user ID 1211, a name 1212, a date of birth 1213, gender 1214, height 1215, weight 1216, an object 1217, and others 1218.

The user ID 1211 is identification information that uniquely specifies the user U2 in the exercise menu management service. The name 1212 is a name of the user. The date of birth 1213 is a date of birth of the user. The current age of the user can be calculated from the date of birth of the user and the current date. The gender 1214 is the gender of the user. The gender 1214 of the user may be omitted as desired by the user. The height 1215 is the height of the user. The weight 1216 is the weight of the user. The object 1217 is user's object of using the exercise menu management service. The object may be an object related to walking such as maintaining health, enhancement of muscles for walking, or improving physical strength, as well as an object for a mind other than the objects related to muscles for walking such as making friends, killing time, or the like.

In the others 1218, for example, information for user authentication, presence or absence of the introduction by existing members, balance of points given when the exercise menu management service is used, a medical history of the user U2, or the like can be recorded.

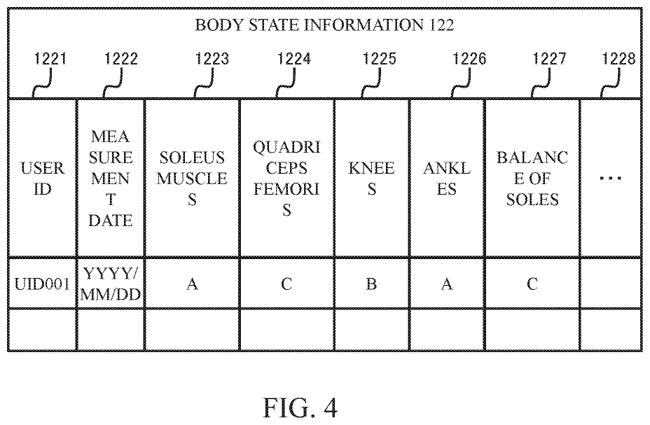

FIG. 4 is an example of the body state information 122. The body state information 122 manages information on the body state of the user U2. Specifically, the body state information 122 manages the body state information (also may be referred to as a walking-related state) related to walking among pieces of information indicating the body state of the user U2.

The body state information 122 includes, for example, a user ID 1221, a measurement date 1222, a state of soleus muscles 1223, a state of a quadriceps femoris 1224, a state of knees 1225, a state of ankles 1226, a state of balance of soles (for example, a distribution of plantar pressure and a locus of a center point of plantar pressure COP) 1227, and others 1228. The term “state” is omitted in the items 1223 to 1227 in the drawing.

In the body state information 122, each state of the soleus muscle, the quadriceps femoris, the knees, the ankles, and the balance of the soles is evaluated by using alphabets such as A to C, for example. “A” indicates a preferable state, “B” indicates a middle state, and “C” indicates a not preferable state. Instead of alphabets, numerals such as 1 to 3, characters such as high, middle, and low may be used.

The user ID 1221 is the same as the user ID 1211 described in FIG. 3. The measurement date 1222 is a date when the body state of the user U2 is measured. The measurement date 1222 may include a time. The state of soleus muscles 1223 to the state of balance of soles 1227 are items corresponding to one example of “a plurality of predetermined parts related to walking among parts of a body of the user U2”.

The items 1223, 1224 are information indicating a state of muscles used for walking. The items 1225, 1226 are information indicating a state of joints used for walking. The item 1227 is information indicating an overall status of walking. The others 1228 is, for example, information indicating a state of parts indirectly related to walking such as a state of a hip joint, a state of muscles of a hip joint (hamstrings, gluteus maximus, gluteus medius, adductor muscle, musculus iliopsoas, or the like), or joints of toes, a state of muscles of toes (plantar muscles, digitorum longus muscles, or the like), a state of muscles of ankles (gastrocnemius muscle, anterior tibial muscle, posterior tibial muscle, peroneus longus muscle, or the like), a state of the abdominal muscle, a state of the spine, or the like. For checking the state of each item, a check method is provided for each item, and the method is, for example, checking of one leg standing for 15 seconds, a distribution of plantar pressure, and a locus of a center point of plantar pressure COP for balance of soles, rock and paper motions of toes for joints and muscles of toes, plantar flexion, dorsiflexion, and twisting to inward/outward for joints and muscles of ankles, a knee bending motion while lying the face down for joints and muscles of knees, knee stretching motion while being seated on a chair, and squats for joints and muscles of knees, and deep squats, split stretch, a hip lift motion for the hip joint.

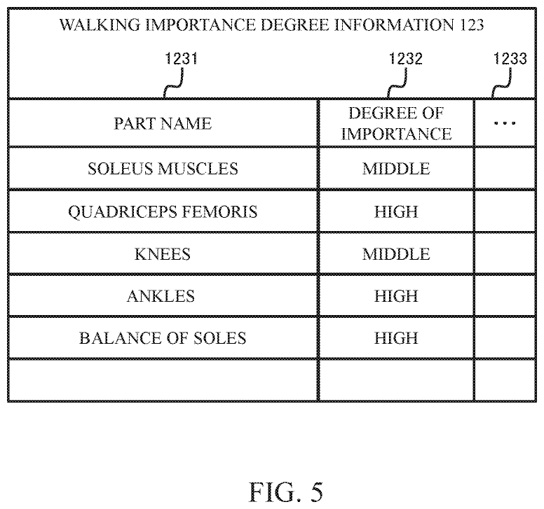

FIG. 5 is an example of the walking importance degree information 123. The walking importance degree information 123 stores a degree of importance related to the walking function for each of parts (a plurality of predetermined parts) related to walking.

The walking importance degree information 123 includes, for example, a part name 1231, a degree of importance 1232, and others 1233. The part name 1231 indicates parts related to walking (soleus muscles, the quadriceps femoris, the knees, the ankles, and the balance of soles, or the like). The degree of importance 1232 indicates a degree of importance of each part related to walking. The others 1233 indicates other information such as remarks, notice, or the like.

In FIG. 5, the degree of importance is indicated by high, middle, and low. “High” indicates a part having a high degree of importance related to walking. “Middle” indicates that a degree of importance related to walking is middle. “Low” (not illustrated) indicates a low degree of importance related to walking. The degree of importance may be evaluated by the two grades of high and middle, or may be evaluated by four or more grades such as 1 to 4.

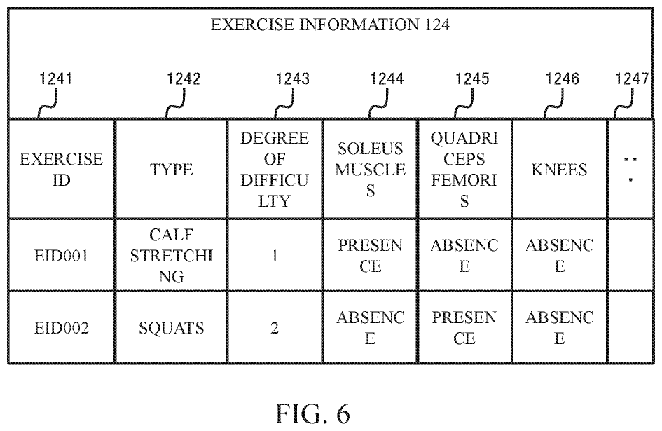

FIG. 6 indicates an example of exercise information 124. The exercise information 124 indicates a relationship between a plurality of exercises contributing to improvement of the walking function and a plurality of predetermined parts.

The exercise information 124 includes, for example, an exercise ID 1241, a type 1242, a degree of difficulty 1243, an effect on soleus muscles 1244, an effect on the quadriceps femoris 1245, an effect on the knees 1246, and others 1247. The effect of “effect on the ankles” and an effect on “the balance of the soles” are not illustrated. The phrase “effect on” is omitted in the items 1244 to 1246 of FIG. 6.

The exercise ID 1241 is information for identifying the exercise. The type 1242 is a type of the exercise. Examples of the type of the exercise include “calf stretching” and “squats”. The degree of difficulty 1243 indicate difficulty when the exercise is performed. The degree of difficulty is represented by numerals such as “1” or “2”. In this example, the exercise is more difficult as the numeral is larger. Regarding the exercise that is difficult to execute, the degree of difficulty of the exercise is a degree of burden or load applied to the user U2 when the exercise is executed.

The effects 1244 to 1246 on each part related to walking indicate whether an effect occurs when the exercise is executed. The exercise related to walking is not effective for all of predetermined parts and sometimes effective only for a specific part, and therefore, the items 1244 to 1246 of effects are provided. “Presence” means that an effect occurs when the exercise is executed. “Absence” means that there is no effect even when the exercise is executed. Evaluation of the effect on predetermined parts is not limited to the presence or absence as described above, and the effect may be evaluated by three or more grades such as 1 to 3, A to C, or high, middle, and low. Moreover, for example, a correction coefficient in consideration of the age, gender, body shape, or the like of the user may be prepared to correct evaluation of the effect.

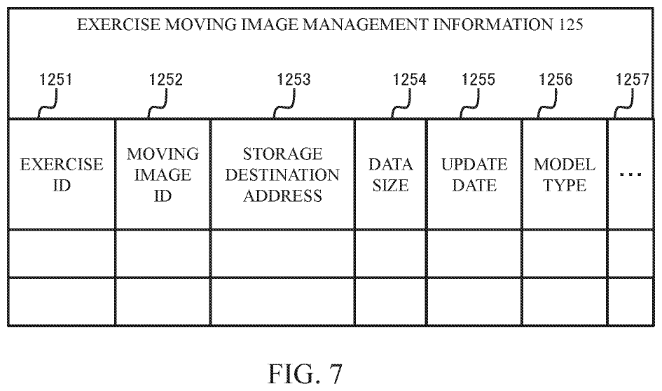

FIG. 7 is an example of the exercise moving image management information 125. The exercise moving image management information 125 relates to the exercise moving image. The exercise moving image management information 125 includes, for example, an exercise ID 1251, a moving image ID 1252, a storage destination address 1253, a data size 1254, an update date 1255, a model type 1256, and others 1257.

The exercise ID 1251 is the same as the exercise ID 1241 in FIG. 6. The moving image ID 1252 is information for identifying the exercise moving image. The exercise moving image is moving image data indicating an example of the exercise by a model. The storage destination address 1253 indicates a place where the data of the exercise moving image is stored. The storage destination of the exercise moving image is not limited to the storage device 102 of the exercise menu management device 1 and may be an external storage system (not illustrated). The data size 1254 is the size of the data of the exercise moving image. The update date 1255 is a creation date or update date of the exercise moving image.

The model type 1256 is a type of a model who presents an example of the exercise. Examples of the type of the model include a male, a female, elderly, middle age, young, slim, muscular, chubby, average, and tall. The type of the model is classified by one or more attributes such as age or body shape as described above. The model type 1256 will be used in the example described later.

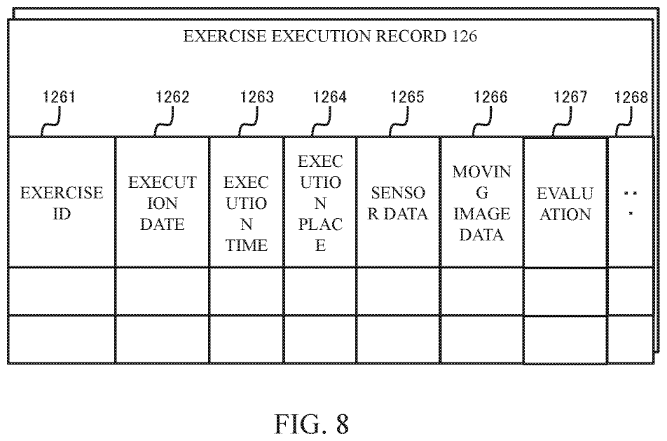

FIG. 8 is an example of the exercise execution record 126. The exercise execution record 126 is a record of execution of the exercise by the user U2. The exercise execution record 126 includes, for example, an exercise ID 1261, an execution date 1262, an execution time 1263, an execution place 1264, sensor data 1265, a moving image data 1266, evaluation 1267, and others 1268.

The exercise ID 1261 is similar to the exercise ID 1251 and the exercise ID 1241. The execution date 1262 is a date when the user U2 executes the exercise. The execution place 1263 is a place where the user U2 executes the exercise. The execution date 1262 and the execution place 1263 may be manually input by the user U2 from the user device 2 to the exercise menu management device 1, or may be automatically input on the basis of information automatically acquired by the exercise menu management device 1 or the user device 2. For example, the time when the user U2 reproduces the exercise moving image or the time when the user U2 ends the reproduction may be recorded as the execution date 1262. A place of the user U2 may be specified by a position information acquisition function (GPS or the like) of the user device 2 and the specified place may be recorded in the execution place 1263.

As the sensor data 1265, sensing data other than a moving image, that is, data measured by the sensor parts 34, 203 during the exercise is recorded. In the sensor data 1265, for example, data of an external environment surrounding the user U2 during the exercise such as a load, pressure, temperature, humidity, or illuminance, and/or vital data such as heartrate, blood pressure, body temperature, amount of perspiration, or complexion of the user U2 during the exercise.

As the moving image data 1266, moving image data obtained by shooting at least a part of the user U2 during the exercise is recorded. The moving image data may be obtained by capturing with any one or both of the camera incorporated in the user device 2 and the external camera connected to the user device 2 (both of the cameras are not illustrated). The external camera may be a fixed camera installed on the ceiling or the desk, or may be a mobile camera mounted in a drone floating in the air or a robot that moves on the floor (both of the cameras are not illustrated).

The evaluation 1267 is evaluation of the exercise executed by the user U2. The evaluation calculated on the basis of the sensor data 1265 and/or the moving image data 1266 is recorded in the evaluation standard 128. For example, by analyzing the sensor data 1265 and the moving image data 1266, it is possible to evaluate as to whether a predetermined amount of load is applied to a predetermined muscle related to walking or whether a joint related to walking moves by a predetermined angle. Artificial intelligence such as a neural network can be used for this evaluation. The system manager U1 can check the exercise moving image and perform evaluation of the exercise moving image.

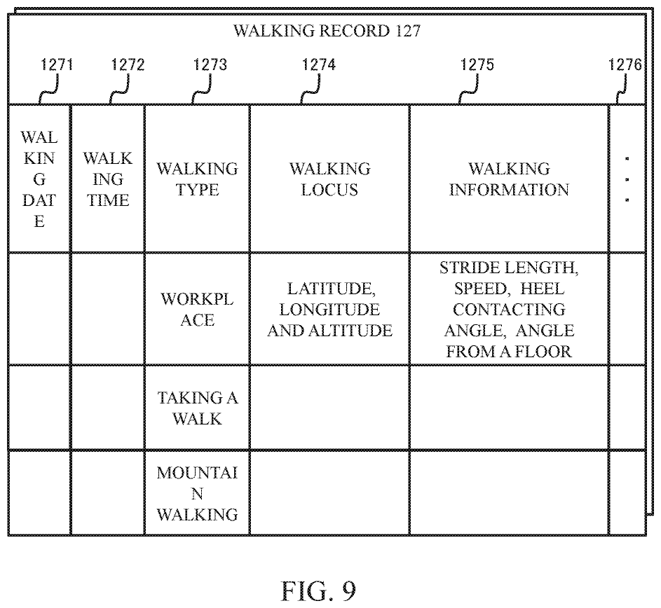

FIG. 9 is an example of the walking record 127. The walking record 127 is a record related to walking at a time other than the time when the user U2 is doing the exercise. Walking other than in the exercise is, for example, commuting to a workplace, commuting to a school, going to a hospital, taking a walk, a walking event, or the like. The walking event will be described later.

The walking record 127 includes, for example, a walking date 1271, a walking time 1272, a walking type 1273, a walking locus 1274, walking information 1275, and others 1276.

The walking date 1271 is a date when the user U2 walks other than in the exercise. The walking time 1272 is a walking time of the user U2. The walking type 1273 is a type of walking such as commuting to a workplace, taking a walk, or mountain walking. The walking locus 1274 is a locus in which the user U2 has walked and includes a plurality of pieces of position information. The position information may include not only coordinates on the map such as latitude and longitude but also altitude. When the sensor part 203 of the user device 2 includes a position information acquisition function such as a GPS and a pressure sensor, a walking record of the user U2 can be detected three-dimensionally.

The walking information 1275 indicates a state of the user U2 at the time of walking. The state at the time of walking is, for example, a stride length, speed, a heel contacting angle, or an angle from a floor. The walking information 1275 may be automatically acquired and recorded, or may be manually inputted. Examples of a method of automatically acquiring the walking information 1275 include analysis of data acquired from a sensor (not illustrated) embedded in a pair of shoes worn by the user U2, and analysis of moving image data from a camera that shoots the feet of the user U2. Examples of a method of manually inputting the walking information 1275 include a method of measuring a stride length, speed, or the like by the user U2 himself/herself and inputting the measured results to the exercise menu management device 1 via the user device 2. Otherwise, there is also a method of visually observing the walking state of the user U2 by an accompanying person and inputting the observed result from the user device 2 held by the accompanying person to the exercise menu management device 1.

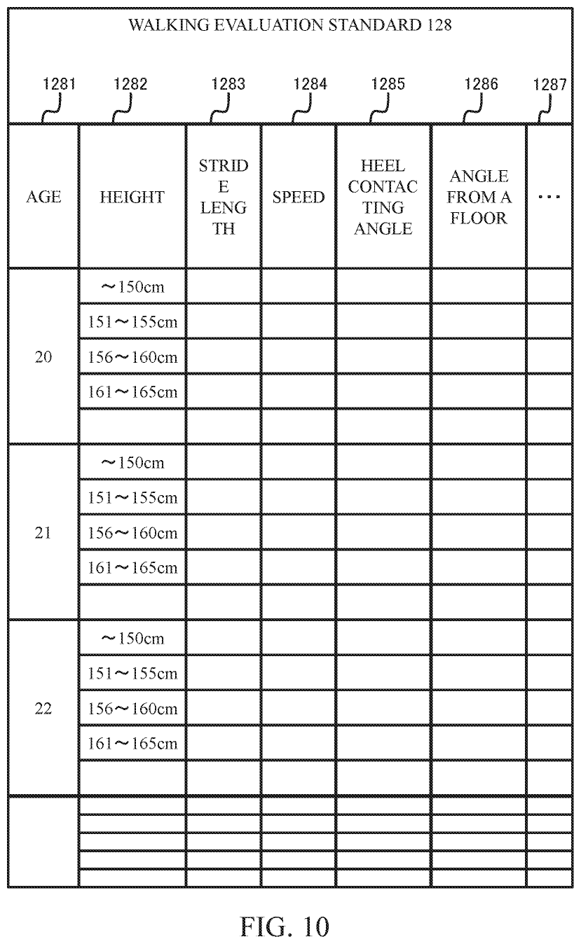

FIG. 10 is an example of the walking evaluation standard 128. The walking evaluation standard 128 is a standard used when the exercise menu management device 1 evaluates the walking record 127 of the user U2.

The walking evaluation standard 128 can be prepared for each user type such as by age, by gender, or by body shape. In FIG. 10, the walking evaluation standard 128 by age and by height is illustrated. The walking evaluation standard 128 includes an age 1281, height 1282, a stride length 1283, speed 1284, a heel contacting angle 1285, an angle from a floor 1286, and others 1287.

The walking evaluation standard 128 illustrated in FIG. 10 has a reference value such as a stride length, speed, heel contacting angle, angle from a floor, or the like by age and by a rank of height.

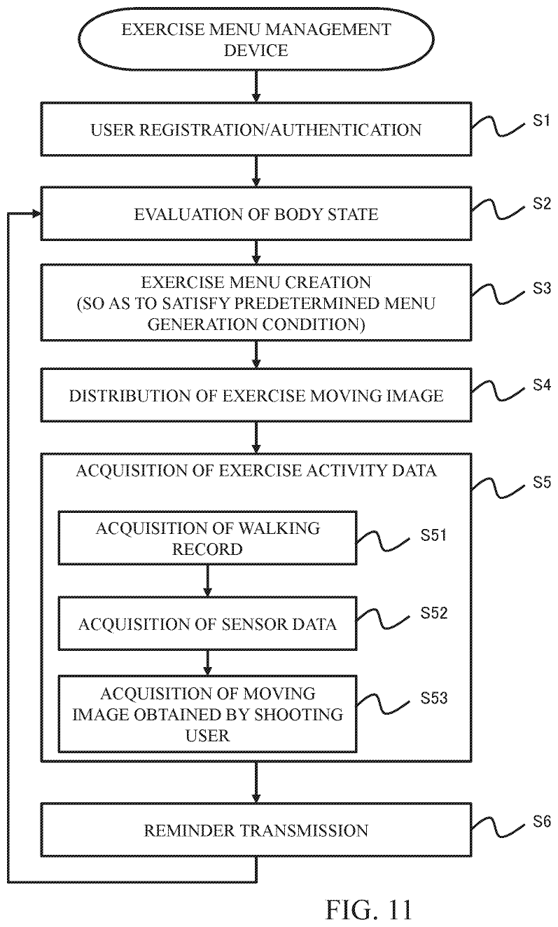

With reference to the flowchart of FIG. 11, the overall operation of the exercise menu management device 1 will be described. Some processing will be described later with reference to other drawings.

The user U2 who attempts to receive a service provided by the exercise menu management system EMS accesses the exercise menu management device 1 by using the user device 2 and performs user registration (S1). The user U2 inputs each item of the user basic information 121 to the exercise menu management device 1 via the user device 2.

When the user U2 whose user registration has been completed accesses the exercise menu management device 1 by using the user device 2, user authentication is performed (S1).

The exercise menu management device 1 evaluates the body state of the user U2 (S2). The exercise menu management device 1 sets evaluation of each item of the body state information 122 described in FIG. 4.

The exercise menu management device 1 creates the exercise menu (S3). That is, the exercise menu management device 1 selects one or more predetermined parts among a plurality of predetermined parts on the basis of the body state information 122 and the walking importance degree information 123, selects a predetermined exercise related to the selected predetermined parts among a plurality of exercises, and creates the exercise menu.

As a case where the state of the quadriceps femoris of the user U2 is not preferable (a case of evaluation C) is described as an example, it can be known that the degree of importance of the quadriceps femoris is large according to the walking importance degree information 123. Accordingly, the exercise menu management device 1 selects an exercise that will contribute to improvement of the state of the quadriceps femoris from exercises registered in the exercise information 124. In this example, squats recognized as being effective for the quadriceps femoris are selected. When there are a plurality of improvement target parts, the exercise menu management device 1 selects an exercise effective for all the plurality of improvement target parts. When there is no exercise effective for all the plurality of improvement target parts, the exercise menu management device 1 selects an exercise effective for a larger number of improvement target parts. As a result, the time consumed for the exercise menu by the user U2 can be made short and the function related to walking of the user U2 can be efficiently improved.

As described above, the exercise menu management device 1 specifies a part to be improved according to the body state of the user U2 and selects an exercise effective for the specified part. A plurality of exercises may be assigned to the part specified as the improvement target. The exercise effective for the part of the improvement target may be selected according to the degree of importance of walking. For example, the exercise menu can be created such that, as the part has a high degree of importance, more exercises effective for the part are selected.

However, an exercise menu including a plurality of exercises having a large degree of difficulty increases a feeling of fatigue of the user U2 and may cause the user U2 to lose the motivation of continuing the exercise. When the exercise menu includes a plurality of exercises having a low degree of difficulty, an amount of time required for completing the exercise menu becomes long and this may increase an overall feeling of fatigue of the user U2.

Then, the exercise menu management device 1 generates the exercise menu so as to satisfy predetermined menu generation conditions described below (S3). For example, the predetermined menu generation conditions are that: (1) functions can be improved in a larger number of improvement target parts as possible with a smaller number of exercises as possible; (2) a degree of fatigue of the user U2 when the exercise menu is completed is equal to or less than a predetermined degree of fatigue; (3) an amount of time required for completion of the exercise menu is equal to or less than a predetermined amount of time required for the exercise; (4) the total degree of difficulty of exercises included in the exercise menu is equal to or less than a predetermined degree of difficulty; and (5) user's posture at the time of using the exercise device 3 has continuity as much as possible.

The degree of fatigue can be calculated from, for example, an amount of time required for completing one exercise, a degree of difficulty set for the exercise, the gender, age, height, weight, a medical history of the user U2. There is no need to use all of the parameters such as the amount of time required for the exercise, the degree of difficulty, and the gender of the user U2, and the degree of fatigue may be calculated from at least one parameter among the parameters. The parameter used for calculating the degree of fatigue may be changed according to the body state information of the user U2, contents of the exercise menu, or the like.

The fact that user's posture at the time of using the exercise device 3 has continuity as much as possible can be defined that, for example, the posture of the user U2 who uses the exercise device 3 does not largely change between the exercises. For example, when the first exercise is executed with the standing posture and the next exercise is executed in the sitting posture, changes in the posture of the user U2 between the exercises are large. By executing the exercise in the standing posture first and then executing the exercise in the sitting posture, the user U2 can reduce the number of times to stand up and sit down.

However, when changes in the posture of the user U2 between the exercises are considered as “hidden exercises” not explicitly indicated in the exercise menu, the condition (5) described above can be rewritten as that the user posture at the time of using the exercise device 3 does not continue as much as possible.

The exercise menu management device 1 transmits a created exercise menu to the user device 2 (S3). The exercise menu management device 1 may transmit the exercise menu to the user device 2 when receiving a request from the user device 2 (S3). The user executes the exercise according to the exercise menu at a desired time or at a time designated by a trainer or the like.

When executing the exercise, the user requests the exercise menu management device 1 to distribute the exercise moving image from the user device 2. Upon receiving the moving image distribution request from the user device 2, the exercise menu management device 1 distributes an exercise moving image corresponding to the requested exercise to the user device 2 (S4). By instructing transmission from the exercise menu management device 1 to a storage system in the outside of the drawing, the exercise moving image may be distributed from the storage system to the user device 2.

The exercise menu management device 1 acquires exercise activity data of the user U2 from the user device 2 regularly or irregularly and manages the data (S5). The exercise activity data is data of execution of the exercise by the user U2 and data of a walking record in an event or the like. The exercise menu management device 1 may acquire data generated along with the exercise activity of the user U2 directly from the exercise device 3. The exercise menu management device 1 can acquire moving image data obtained by shooting the user U2 doing the exercise with a camera (not illustrated) provided in a space where the user U2 does the exercise.

Step S5 of acquiring the exercise activity data includes, for example, step S51 of acquiring a walking record, step S52 of acquiring sensor data, and step S53 of acquiring a moving image obtained by shooting the user.

In step 51 of acquiring a walking record, data related to walking at the time of commuting, the time of taking a walk, or the like of the user U2 is acquired from the user device 2 or a sensor (not illustrated) attached to the user U2, and the data is recorded in the walking record 127. In step S52 of acquiring sensor data, sensor data is acquired from the user device 2 or the exercise device 3, and the sensor data is recorded in a sensor data column 1265 of the exercise execution record 126. In step S53 of acquiring a moving image obtained by shooting the user, moving image data obtained by shooting the user U2 during the exercise is acquired from a camera connected to the user device 2 or a camera provided in a space where the user U2 is doing the exercise, and the moving image data is recorded in a moving image data column 1266 of the exercise execution record 126.

Steps S51 to S53 are not performed continuously but are performed at a timing when acquisition is possible. For example, when walking of the user U2 is detected, the record of the walking is acquired and recorded (S51). When the user U2 executes the exercise at another timing, sensor data and a moving image of the user U2 during the exercise are acquired and recorded (S52, S53).

The exercise menu management device 1 refers to the execution date 1262 of the exercise execution record 126. When the exercise menu management device 1 finds a user U2 whose last time of exercise execution is a predetermined time or more ago, the exercise menu management device 1 transmits a reminder to the user device 2 of the user U2 (S6). The reminder can be made by, for example, an email, a short message, synthesized sound, vibration, and a combination of these.

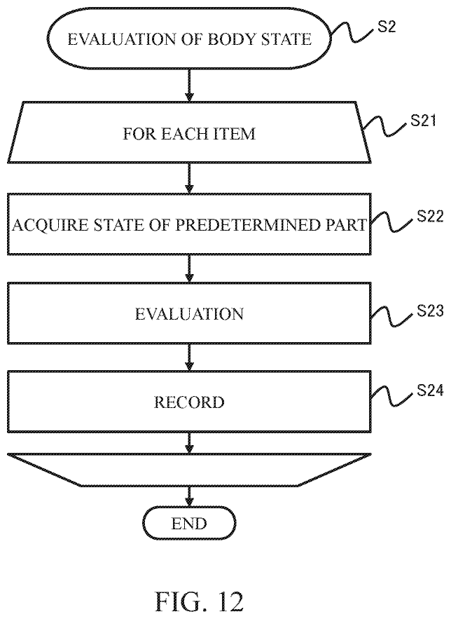

FIG. 12 is a flowchart illustrating a detail of processing (step S2 of FIG. 11) of evaluating a body state of the user. The exercise menu management device 1 performs steps S22 to S24 as below for each of items 1223 to 1227 of the body state information 122, that is, for each of a plurality of predetermined parts (S21) related to walking among the main body parts of the user U2.

The exercise menu management device 1 acquires a state of a predetermined part being a target (S22), evaluates the acquired state of the predetermined part (S23), and records the evaluation result in a corresponding item of the body state information 122 (S24).

The state of a predetermined part of the user U2 may be measured by a trainer in a sport gym, or, as the example described later, a result of measurement by the user U2 as a self-check may be transmitted to the exercise menu management device 1. The evaluation as to the state of the predetermined part may be determined by a trainer, may be determined by using artificial intelligence such as a neural network, or may be determined by a trainer with reference to a determination result by artificial intelligence. The execution timings of steps S22, S23, S24 do not need to be continuous.

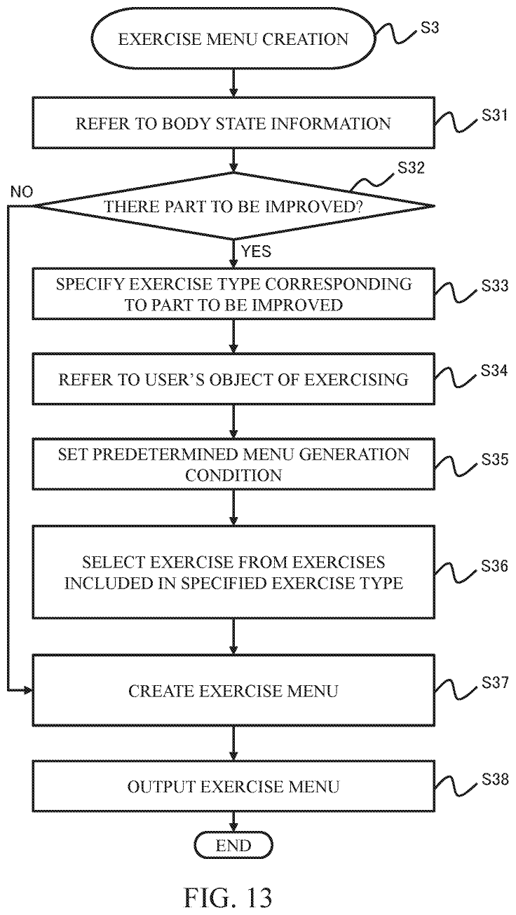

FIG. 13 is a flowchart illustrating details of exercise menu creation processing (step S3 of FIG. 11). The exercise menu management device 1 refers to the body state information 122 of the target user U2 (S31), and determines whether there is a part to be improved among the predetermined parts of the user U2 related to walking (S32). When there is no part to be improved (S32: NO), the process proceeds to step S37 described later.

When there is a part to be improved (S32: YES), the exercise menu management device 1 specifies an exercise type (stretch, squats, or the like) effective for the part to be improved (S33).

Moreover, the exercise menu management device 1 refers to the user basic information 121, checks an object of the user of exercising (S34), and sets a menu generation condition suitable for the object of the user (S35). User's object of exercising can be changed as needed. The exercise menu management device 1 selects an exercise effective for the part to be improved on the basis of the object of the user and the predetermined menu generation condition (S36).

For example, when the object of the user is actively maintaining or improving the walking function such as maintaining health or enhancing walking muscles of walking, the exercise menu management device 1 selects an exercise such that only exercises effective for the part to be improved are efficiently performed. On the contrary, for example, when the object of the user is making friends or killing time, the exercise menu management device 1 selects not only exercises effective for the part to be improved but also exercises having a low degree of difficulty and exercises having a low degree of fatigue.

The exercise menu management device 1 creates an exercise menu on the basis of the exercise selected in step S36 (S37). The exercise menu indicates an execution order of the selected exercises, and in addition, includes link information for reproducing the exercise moving image being an example of the selected exercises. The exercise menu management device 1 transmits the created exercise menu to the user device 2 (S38). Otherwise, the exercise menu management device 1 stores the created exercise menu and waits for a transfer request from the user device 2.

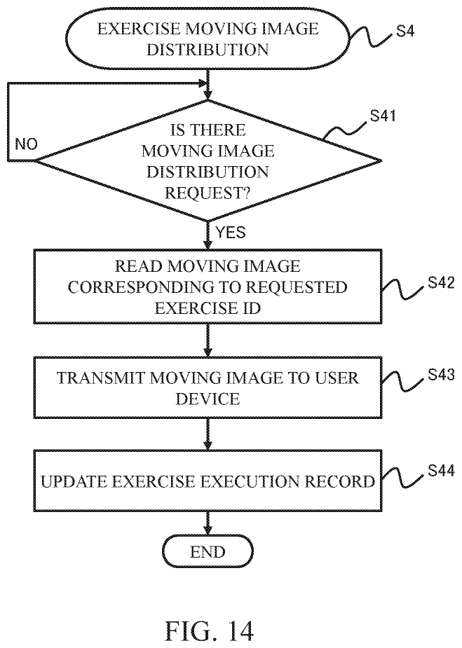

FIG. 14 is a flowchart illustrating details of processing of distributing an exercise moving image (step S4 of FIG. 11).

Upon receiving a moving image distribution request from the user device 2 (S41: YES), the exercise menu management device 1 reads the exercise moving image corresponding to the requested exercise ID (S42) and transmits the read exercise moving image to the user device 2 (S43). The exercise menu management device 1 considers that the exercise moving image has been reproduced in the user device 2 and the user U2 has executed the exercise, and sets a transmission end time of the exercise moving image to the execution date 1262 of the exercise execution record 126 of the user U2 (S44). The transmission start time of the exercise moving image may be set as the execution date 1262. It is sufficient that the reproduction time of the exercise moving image is set to the execution time 1263 of the exercise execution record 126.

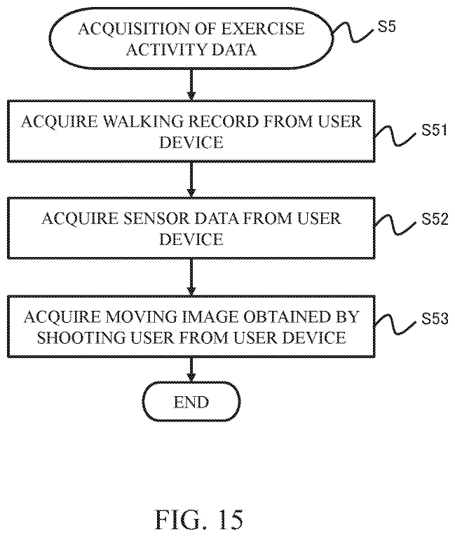

The flowchart of FIG. 15 illustrates details of processing of acquiring exercise activity data (step S5 in FIG. 11). As described above, the exercise menu management device 1 acquires a walking record from the user device 2 and stores the walking record in the walking record 127 (S51). The exercise menu management device 1 acquires sensor data from the user device 2, and stores the sensor data in the sensor data column 1265 of the exercise execution record 126. Moreover, the exercise menu management device 1 acquires a moving image data obtained by shooting the user from the user device 2, and stores the moving image data in the moving image data column 1266 of the exercise execution record 126 (S53).

It is sufficient that sensor data in a time zone corresponding to the reproduction time of the exercise moving image among pieces of sensor data stored in the user device 2 is extracted as the sensor data at the time of the exercise. The reproduction time of the exercise moving image can be obtained by setting the transmission start time (or transmission end time) of the exercise moving image as the reproduction start time, and setting the time obtained by adding the reproduction required time of the exercise moving image to the start time as the reproduction end time. As described above, the sensor data at the time of the exercise may be extracted from the pieces of sensor data in the user device 2 by using artificial intelligence such as a neural network, instead of the method of extracting the sensor data by specifying the time when the exercise moving image is reproduced.

The moving image data obtained by shooting the user can be obtained from not only the camera incorporated in the user device 2 but also any one or more of the external cameras connected to the user device 2 via a wire or wirelessly and the cameras installed in the space where the user U2 does the exercise.

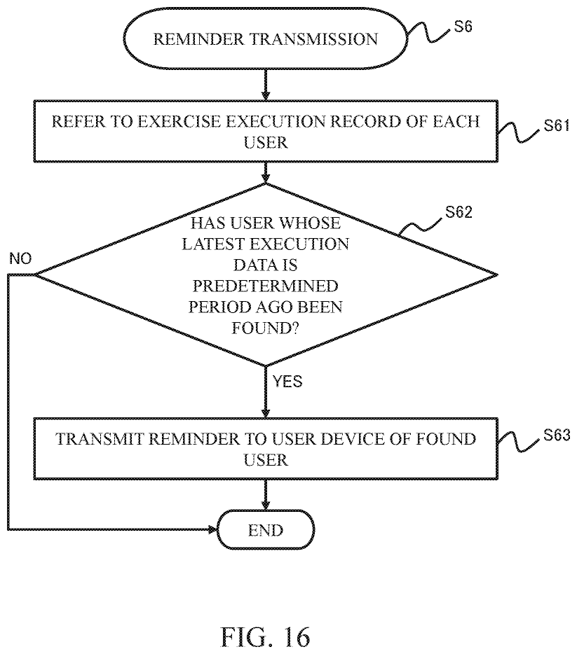

The flowchart of FIG. 16 illustrates details of reminder transmission processing (step S6 in FIG. 11). The exercise menu management device 1 refers to the exercise execution record 126 of each user U2 (S61), and detects the user U2 whose latest exercise execution date is a predetermined period ago (S62). When the exercise menu management device 1 finds the user U2 who has not done an exercise for a predetermined period or more (S62: YES), the exercise menu management device 1 transmits a reminder to the user device 2 of the found user U2 (S63).

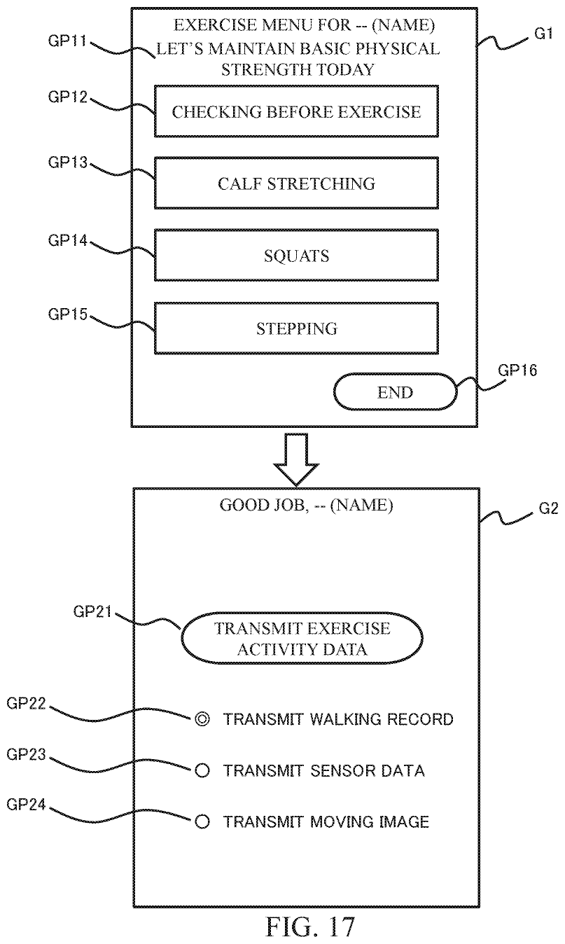

FIG. 17 is an example of a screen provided from the exercise menu management device 1 to the user device 2. A screen G1 illustrated in the upper side of FIG. 17 displays the exercise menu received from the exercise menu management device 1.

The exercise menu screen G1 includes, for example, an encouragement message part GP 11 that displays an encouraging message, exercise buttons GP 12 to GP 15 corresponding to the selected exercise, and a button GP 16 for closing the screen. The exercise buttons GP12 to GP15 also serve as buttons for instructing reproduction of the exercise moving image. When the user U2 operates the exercise button, the exercise moving image corresponding to the operated button is transferred from the exercise menu management device 1 to the user device 2 and the exercise moving image is automatically reproduced.

The exercise buttons GP12 to GP15 are arranged in the actual order. The user U2 reproduces the exercise moving images from the top in order and executes the exercises. The exercise buttons can be set such that the exercise buttons cannot be operated in the order different from a predetermined order in the exercise menu. However, a configuration may be adopted in which, in the exercise menu, the execution order of the exercises is not determined and the exercise buttons can be operated in an order desired by the user U2.

The lower side of FIG. 17 illustrates a screen G2 displayed in the user device 2 when the exercise ends. The exercise end screen G2 includes a button GP21 for transmitting the exercise activity data to the exercise menu management device 1, and radio buttons GP22 to GP24 for specifying the contents of data to be transmitted.

At the time of transmitting the exercise activity data from the user device 2 to the exercise menu management device 1, the user U2 can determine which is included in the data to be transmitted among the walking record (GP22), the sensor data (GP23), and the moving image (GP24) in which the user appears. The initial value may be set such that all of the walking record, the sensor data, and the moving image are transmitted. Otherwise, the initial value may be set such that nothing is transmitted.

According to this example configured as described above, an exercise menu useful for improving the walking function of the user U2 can be created on the basis of the body state of the user U2 and provided to the user U2, so that the walking function of the user can be improved to achieve healthy longevity.

According to this example, the user U2 can execute an exercise by using the user device 2 and the exercise device 3, and the result of execution of the exercise by the user is recorded in the exercise menu management device 1. Accordingly, the user U2 and the trainer U1 can easily check the execution status of the exercises by referring to the exercise execution record 126.

### Example 2

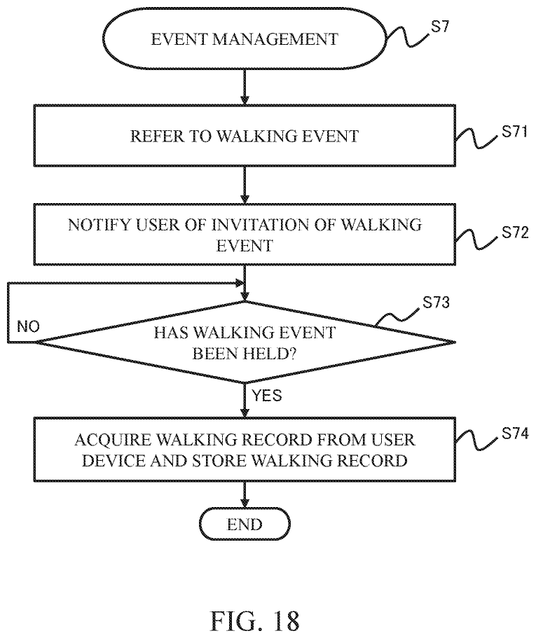

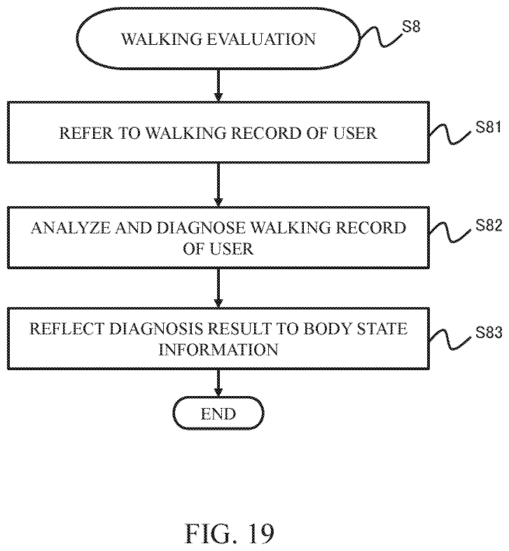

Example 2 will be described with reference to FIGS. 18 and 19. In each of the following Examples including this example, differences from Example 1 will be mainly described. In this example, an event related to walking is provided to the user U2, and effects by the event are measured and the results are used for creating exercise menus.

FIG. 18 is a flowchart illustrating event management processing S7. The exercise manager U1 such as a trainer can register walking event information in the storage device 102 of the exercise menu management device 1. Although not illustrated, the walking event information includes items of, for example, an event ID, an event name, a scheduled date and time of the event, a place of the event, contents of the event, presence or absence of execution of the event, a name of a person in charge, an ID of a user who plans to participate in the event, and others.

The walking event is, for example, an event in which walking by the participant is expected, such as mountain walking, hiking, mountain climbing, taking a walk, garden party, going to see town sights, sightseeing, cherry blossom viewing, dancing, or bon dancing. In the walking event, the means of transport other than walking such as a bus, an automobile, a taxi, a train, an airplane, a ship, a gondola, or a lift can be used.

The event management part 14 of the exercise menu management device 1 refers to a registered walking event regularly or irregularly (S71). The event management part 14 compares the scheduled date and time of the registered walking event and the current date and time, and when the difference between the scheduled date and time of the walking event and the current date and time become equal a predetermined amount of time, the event management part 14 transmits an invitation for the walking event to the user U2 (S72).

The invitation of the walking event is transmitted to the user U2 as, for example, an electronic invitation such as an email, a short message, or a sound message. However, the invitation is not limited to the electronic invitation, and a paper invitation may be posted or a trainer may invite the user U2 by phone call.

The event management part 14 compares the current date and time and the scheduled date and time of the walking event, and determines whether the walking event has been held (S73). When the event management part 14 determines that the walking event has been held (S73: YES), the event management part 14 acquires a walking record from the user device 2 and stores the walking record in the walking record 127 (S74). When the date and time in which the walking event actually held is stored in the walking event information, the event management part 14 checks the date and time in which the walking event actually held, and then requests the user device 2 to transmit the walking record.

FIG. 19 is a flowchart of processing S8 of evaluating the walking record. The walking evaluation part 15 refers to the walking record 127 acquired from the user device 2 (S81), analyzes and diagnoses the walking record (S82), and reflects the diagnosis result to each of the items 1223 to 1227 of the body state information 122 (S83).

The walking evaluation part 15 calculates a state of a predetermined part related to walking from sensor data and/or moving image data obtained by shooting the user, compares the calculation result and the walking evaluation standard 128, and diagnoses the state of the predetermined part.

This example configured as described above also exhibits similar operation and effect to those of Example 1. Also in this example, an event accompanied by walking is provided to the user U2, and the state of the predetermined part of the user U2 who has participated in the event is automatically detected and reflected in the body state information 122, so that the user U2 can improve his/her walking function while enjoying it.

### Example 3

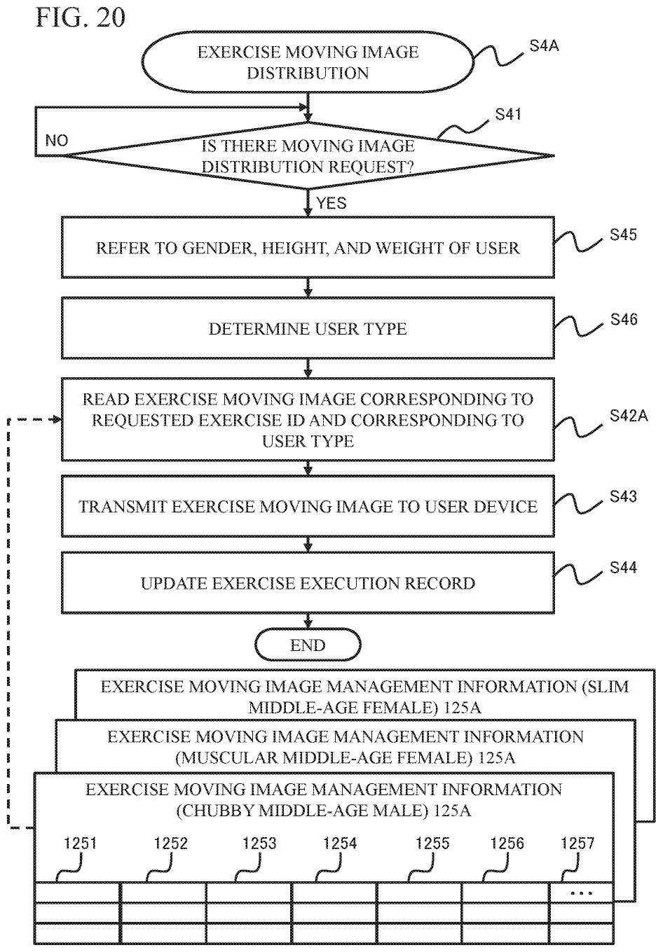

Example 3 will be described with reference to FIG. 20. In this example, an exercise moving image according to the type of the user U2 is distributed to the user device 2. The flowchart of FIG. 20 illustrates processing S4A of distributing an exercise moving image.

When the exercise menu management device 1 receives a moving image distribution request (S41), the exercise menu management device 1 refers to the gender, height, and weight of the user U2 (S45), and determines the type of the user U2 from these pieces of information (S46). The user type is prepared in advance on the basis of an attribute of the user, and classified as, for example, a “chubby middle-age male”, a “muscular middle-age female”, a “slim middle-age female”, or the like. User types other than these may be included. Gender may be removed from the user type.

The exercise menu management device 1 reads an exercise moving image that corresponds to the requested exercise ID as well as to the user type (S42A), and transmits the read exercise moving image to the user device 2 (S43). The exercise menu management device 1 updates the exercise execution record 126 (S44).

As illustrated in the lower side of FIG. 20, in this example, an exercise moving image is prepared for each user type in advance. The exercise moving images for each user type are moving images in which a type of a model (trainer) who shows an example of an exercise is the same as the user. That is, an exercise moving image in which a model classified as a chubby middle-age male appears is provided to a user U2 who is a chubby middle-age male.

This example configured as described above also exhibits similar operation and effect to those of Example 1. In this example, an exercise moving image in which a model corresponding to the type of the user U2 appears is provided, so that the user U2 is easy to copy the motion of the example and usability for the user U2 is further improved.

### Example 4

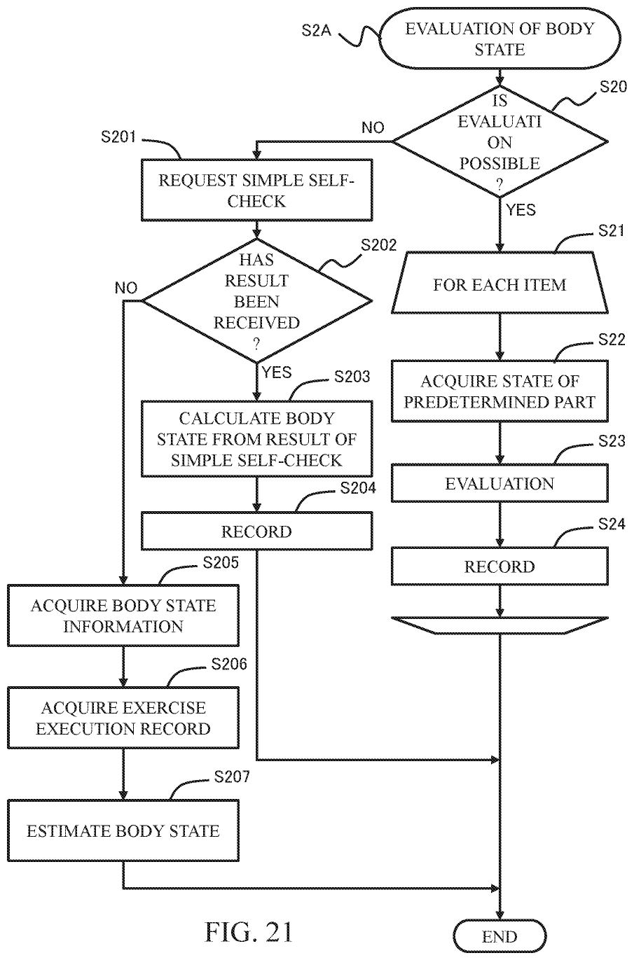

Example 4 will be described with reference to FIG. 21. In this example, a modification of the evaluation method of the body information will be described. FIG. 21 is a flowchart of processing of evaluating the body state.

The exercise menu management device 1 determines whether the body state can be evaluated (S20). When the body state information of the user U2 can be acquired, the body state can be evaluated as described in FIG. 12 (S20: YES, S21 to S24).

On the contrary, for example, when the user U2 is during travel or a business trip and is in a place where no installation for measuring the body state exists, the body state of the user U2 cannot be acquired (S20: NO). Then, the exercise menu management device 1 request the user U2 to perform a simple self-check (S201). The simple self-check is checking the body state of the user U2 by the user U2 himself/herself. The result of the simple self-check is transmitted from the user device 2 to the exercise menu management device 1.

For example, text or a moving image of explanation of a method of the simple self-check is transmitted from the exercise menu management device 1 to the user device 2. The user U2 reads the explanation text or watches the explanation moving image, checks the state of the muscles, joints, or the like of himself/herself, and transmits the result from the user device 2 to the exercise menu management device 1.

The exercise menu management device 1 determines whether the result of the simple self-check has been received from the user device 2 (S202). When the exercise menu management device 1 receives the result of the simple self-check from the user device 2 (S202: YES), the exercise menu management device 1 evaluates the body state of the user U2 on the basis of the result of the simple self-check (S203), and records the body state in the body state information 122 (S204).

When the exercise menu management device 1 cannot receive the result of the simple self-check from the user device 2 (S202: NO), the exercise menu management device 1 acquires the body state information 122 (S205) and further acquires the exercise execution record 126 (S206). Then, the exercise menu management device 1 evaluates the current body state of the user U2 on the basis of the body state information 122 and the exercise execution record 126 recorded at the last time (S207). The current body state of the user U2 can be evaluated to some extent on the basis of the exercise execution record 126 that has been performed after the latest evaluation of the body state. Artificial intelligence such as a neural network can be used for this evaluation.

This example configured as described above also exhibits similar operation and effect to those of Example 1. In this example, even when the body state of the user U2 cannot be evaluated at the exercise menu creation base ST1 due to a business trip, travel, or the like of the user, the body state of the user U2 can be evaluated by estimation based on the result of the simple self-check or latest data 122, 126.

The exercise menu management device 1 can create an exercise menu suitable for the body state of the user U2 with the estimation values based on the result of the simple self-check or the latest data 122, 126, and provide the exercise menu to the user U2.

As a result, the user U2 can obtain the exercise menu suitable for evaluation of the body state of himself/herself even without going to the exercise menu creation base ST1, which improves usability.

### Example 5

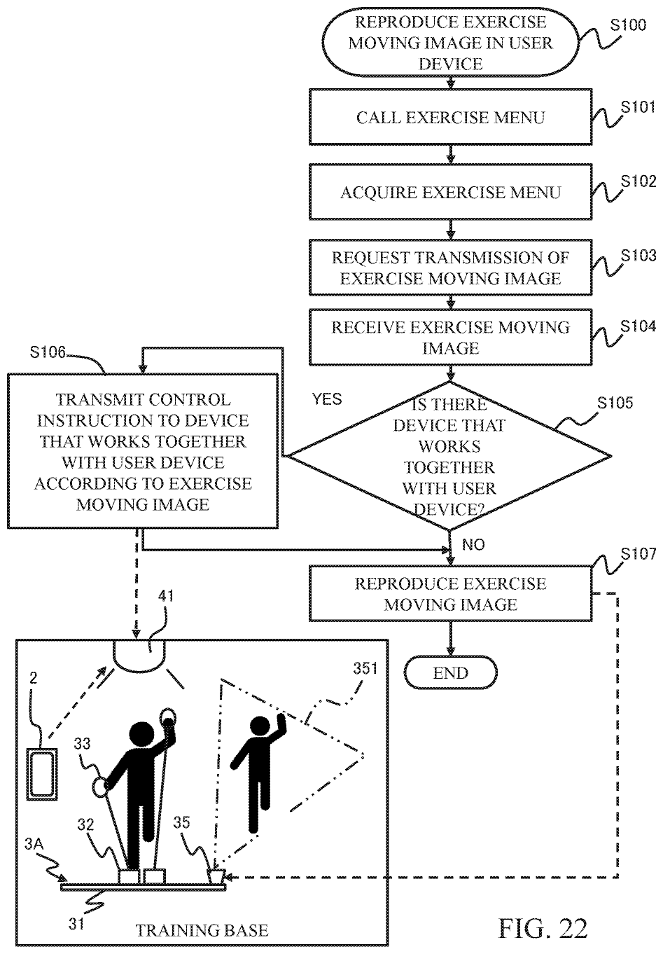

Example 5 will be described with reference to FIG. 22. In this example, the user device 2 works together with an electronic device 41 external to the user device 2 at the time of reproducing the exercise moving image. Moreover, the exercise menu management device 3A of this example includes a hologram projection device 35 as an example of the “information provision part”.

FIG. 22 is a flowchart illustrating processing of reproducing an exercise moving image in the user device 2. When the user device 2 accesses the exercise menu management device 1 and logins in the exercise menu management service, the exercise menu for the user U2 is called (S101).

The user device 2 acquires the exercise menu from the exercise menu management device 1 (S102), and waits for a reproduction instruction from the user U2. When the user U2 instructs to reproduce an exercise moving image, the user device 2 requests the exercise menu management device 1 to transmit the exercise moving image specified by the user U2 to the user device 2 (S103).

When receiving the exercise moving image (S104), the user device 2 determines whether there are devices that work together with the user device 2 at the time of reproduction of the exercise moving image, in the periphery of the user device 2 (S105). For example, there is an electronic device 41 capable of working together with the user device 2 among electronic products such as a lighting device, a television device, a speaker, an automatic vacuum cleaner, or a nursing care robot within a predetermined range from the user device 2 (S105: YES), the user device 2 transmits a control instruction to the electronic device 41 (S106).

The control instruction is, for example, an instruction to operate the electronic device 41. When the electronic device 41 is a television device, the user device 2 causes the television device to reproduce a cheering message of text, sound, or a moving image. When the electronic device 41 is a lighting device, the user device 2 transmits an instruction to the electronic device 41 to blink a light. When the electronic device 41 is a speaker, the user device 2 transmits sound data to the electronic device 41 and causes the electronic device 41 to reproduce the sound data. The sound data is, for example, the sound of applause, sound of an instrument, or yells. When the electronic device 41 is an automatic vacuum cleaner, the user device 2 operates or stops the vacuum cleaner. When the electronic device 41 is a nursing care robot, the user device 2 operates at least a part of a movable part of the nursing care robot, causes sounds to be outputted, or causes a lamp or a display to blink.

After the user device 2 transmits the control instruction to the electronic device 41, the user device 2 reproduces the exercise moving image (S107). On the contrary, when there is no electronic device 41 that works together with the user device 2 in the periphery of the user device 2 (S105: NO), the user device 2 reproduces the exercise moving image (S107).

The exercise moving image may be displayed on a terminal screen of the user device 2, or may be projected on the television device as described in FIG. 1. Moreover, the exercise device 3A of this example includes the hologram projection device 35. The hologram projection device 35 projects a three-dimensional hologram 351 of the exercise moving image received from the user device 2 as an example of the “information related to the exercise”.

This example configured as described above also exhibits similar operation and effect to those of Example 1. Moreover, in this example, since the user device 2 can work together with the electronic device 41 existing in the periphery of the user device 2 to reproduce the exercise moving image, the user U2 can execute the exercise in a fun environment. Moreover, in this example, since the hologram projection device 35 is provided in the exercise device 3A, the user U2 can three-dimensionally check the motion of the model as an example, and realistic feeling is enhanced. The hologram projection device 35 may be connected to the user device 2.

### Example 6

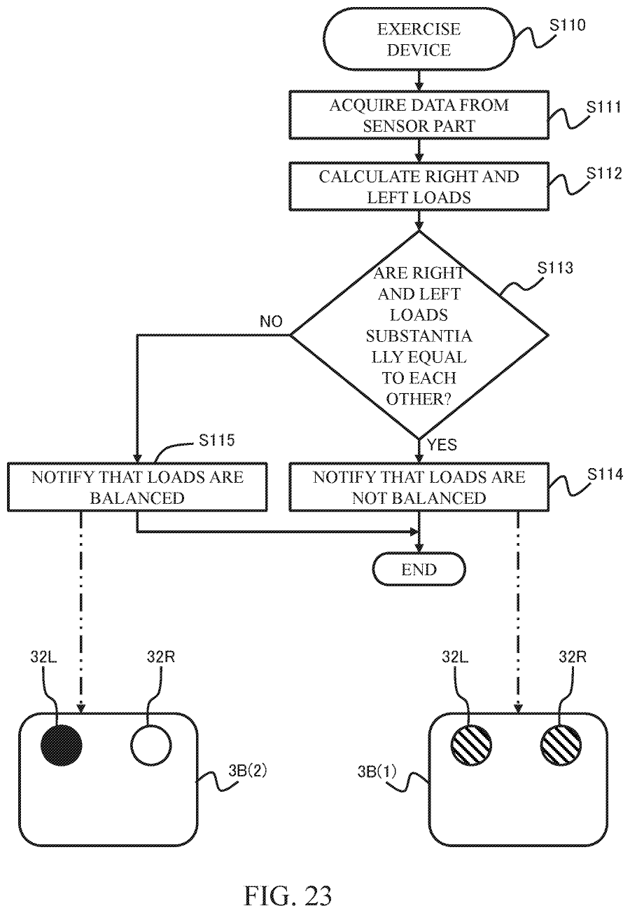

Example 6 will be described with reference to FIG. 23. In an exercise device 3B of this example includes main body parts 32L, 32R each including a light as the “information provision part”. The main body parts 32L, 32R including the light actuates the light according to a detected load, for example. Here, an example will be described in which the lights are caused to blink according to a sole balance of the user U2.

FIG. 23 is a flowchart illustrating processing performed by the exercise device 3B. The exercise device 3B acquires data from the sensor part 34 (S111) and calculates right and left loads (S112). The exercise device 3B determines whether the right and left loads are substantially equal to each other (S113). When the exercise device 3B determines that the right and left loads are substantially equal to each other (S113: YES), the lights of the right and left main body parts 32R, 32L are turned on in the same manner, thereby notifying the user U2 of the fact that the right and left soles are balanced (S114). On the contrary, when the exercise device 3B determines that the right and left loads are not substantially equal to each other (S113: NO), the lights of the right and left main body parts 32R, 32L are turned on in different manners, thereby notifying the user U2 of the fact that the right and left soles are not balanced (S115).

In the exercise device 3B(1) illustrated in the lower right of FIG. 23, the lighting state of the light when the right and left loads are balanced is illustrated. In the exercise device 3B(2) illustrated in the lower left of FIG. 23, the lighting state of the light when the right and left loads are not balanced is illustrated.

This example configured as described above also exhibits similar operation and effect to those of Example 1. Moreover, in this example, the lights are provided in the main body parts 32L, 32R of the exercise device 3B, and turned on in a manner of working together with each other according to the state of the loads as the “information related to the exercise” measured during the exercise. Accordingly, the user U2 can easily check whether the right and left loads are balanced during the exercise.

When the user U2 does the exercise by using only one of the two main body parts 32L, 32R, the right and left loads are not balanced and the lighting state is as described in the lower left of FIG. 23. However, the user U2 is aware that the user U2 is doing the exercise in which the right and left loads are not balanced and the lighting state does not affect the user U2.

### Example 7

Example 7 will be described with reference to FIG. 24. In this example, the user U2 can appropriately use a plurality of training bases ST2 and a plurality of exercise menu creation bases ST1. Moreover, in this example, the user U2 can use one or more check bases ST3 to measure the body state of the user U2 himself/herself.

Example 1 describes the case where the user U2 obtains the exercise menu suitable for the body state in the exercise menu creation base ST1 and executes the exercise in the training base ST2 such as a home. On the contrary, in the exercise menu management system EMS of this example, the user U2 can receive an exercise menu suitable for the body state of the user U2 himself/herself from an exercise menu management device 1 in any one of the plurality of exercise menu creation bases ST1. Then, the user U2 can do the exercise based on the exercise menu in any one or more of the plurality of training bases ST2.

Moreover, in the exercise menu management system EMS of this example, at least one check base ST3 can be provided. The check base ST3 is provided with a body state check device 5 for measuring the body state of the user U2.

For example, the user U2 usually have an exercise menu created in the exercise menu creation base ST1 near a workplace and executes the exercise in the training base ST2 such as a home. When the user U2 is away from the home for a long time due to a business trip, travel, or the like, the user U2 can execute the exercise in the accommodation, station, airport, ferry, or the like as a temporary training base ST2. In a case of a business trip or travel for a long time, the user U2 can have the latest exercise menu created in the exercise menu creation base ST1 at the destination of the business trip or travel.

Moreover, the user U2 can measure the body state of the user U2 himself/herself in the check base ST3 installed in, for example, an airport, station, hotel, department store, sport goods store, or book store, receive the measurement result by the user device 2, and cause the user device 2 to transmit the measurement result to any one of the exercise menu management devices 1. The plurality of the exercise menu management devices 1 are communicably connected to each other and can refer to each piece of data associated with the user ID, with each other. Otherwise, a configuration may be adopted in which the storage part 12 illustrated in FIG. 1 is provided in an external file storage (not illustrated), and the exercise menu management devices 1 share pieces of data 121 to 128 of the user U2.

This example configured as described above also exhibits similar operation and effect to those of Example 1. Moreover, in this example, since the user U2 can execute the exercise by using the plurality of exercise menu creation bases ST1 and the plurality of training bases ST2, even in a case of going out due to a business trip or travel, the user U2 can continue the exercise at the destination, so that health of a predetermined part related to walking of the user U2 can be appropriately maintained.

The present disclosure is not limited to the examples described above and includes various modifications. For example, the examples described above are described in detail for explanation of the present disclosure so as to be easy to understand, and the present disclosure is not limited to an example including all of the described configurations. Some of configurations of one example can be replaced with configurations of another example, or a configuration of another example can be added to a configuration of one example. Regarding some of configuration of each example, addition, deletion, and replacement by other configurations are possible. The examples can be combined as appropriate unless it is an obvious contradiction.

The output part can output the exercise menu to a user device used by the user.

The exercise information can further include a degree of difficulty when the plurality of exercises are performed, and the exercise menu creation part can select the predetermined exercise on the basis of the degree of difficulty from among exercises related to the predetermined parts that has been selected from among the plurality of exercises.

The exercise menu creation device may further include an event management part that acquires data related to walking of the user and manages the data when the user participates in an event contributing to improvement of the walking function.

The user device further includes a communication part that communicates with an exercise device used by the user, and the exercise device can include a sensor part that detects information of time when the user performs an exercise, and an information management part that manages the information detected by the sensor part and transmits the information to the communication part.

The exercise device can further include an information provision part, and the information provision part provides the user with information related to the exercise.

The exercise device may include a board part placed on a floor, a plurality of body parts provided on the board part, and an attachment part provided in each of the main body parts so as to be expandable and attached to the body of the user, each of the main body parts exerts a force of pulling the attachment part separated from the main body parts to the main body parts, the information provision part is provided in each of the main body parts, and each of the information provision part may provide the user with the information related to the exercise.

The output part can output an exercise menu to the user device, cause a moving image to be read from an exercise moving image management information storage part that manages the moving image related to the exercise menu, and cause the moving image to be distributed to the user device.

A predetermined moving image is prepared according to a type of the user, and the output part may cause the moving image according to the type of the user to be read from an exercise moving image management information storage part and cause the moving image to be distributed to the user device.

An exercise menu management method according to another aspect of the present disclosure is a method of creating an exercise menu related to improvement of a walking function by an exercise menu management device and providing a user with the exercise menu, the exercise menu management device stores body state information being information of a plurality of predetermined parts related to the walking function among parts of a body of the user, stores walking importance degree information indicating a degree of importance related to the walking function for each of the plurality of predetermined parts, stores exercise information indicating a relationship between a plurality of exercises contributing to improvement of the walking function and the plurality of predetermined parts, selects one or more of the plurality of predetermined parts on the basis of the body state information and the walking importance degree information, creates the exercise menu by selecting a predetermined exercise related to the predetermined parts selected from a plurality of the exercises, and outputs the exercise menu that has been created.

A computer program according to still another aspect of the present disclosure causes a computer to execute steps of: storing body state information being information of a plurality of predetermined parts related to the walking function among parts of a body of the user; storing walking importance degree information indicating a degree of importance related to the walking function for each of the plurality of predetermined parts; storing exercise information indicating a relationship between a plurality of exercises contributing to improvement of the walking function and the plurality of predetermined parts; selecting one or more of the plurality of predetermined parts on the basis of the body state information and the walking importance degree information; creating the exercise menu by selecting a predetermined exercise related to the predetermined parts selected from a plurality of the exercises; and outputting the exercise menu that has been created.

The present disclosure includes, for example, the embodiment(s) that can be expressed as below.

Expression 1: An exercise menu management device in which an output part outputs an exercise menu to a user device, causes a moving image to be read from an exercise moving image management information storage part that manages the moving image related to the exercise menu, and causes the moving image to be distributed to the user device.

Expression 2: The exercise menu management device according to Expression 1, in which the moving image is prepared according to a type of the user, and the output part causes the moving image according to the type of the user to be read from the exercise moving image management information storage part and causes the moving image to be distributed to the user device.

Expression 3: The exercise menu management device according to Expression 2, in which, when the user device reproduces the moving image related to the exercise menu, the user device works together with an electronic device external to the user device.

Expression 4: The exercise menu management device according to Expression 1, further including a user management part that manages an execution status of the exercise menu by the user, in which the output part causes the moving image related to the exercise menu to be read from the exercise moving image management information storage part and distributed to the user device in response to a request from the user device, and, when the moving image related to the exercise menu is distributed to the user device, the user management part determines that at least a part of the exercise menu has been executed by the user.

Expression 5: The exercise menu management device according to Expression 4, further including a user interface device for manager that is used by a manager who manages the exercise of the user, in which the body state information is measured in an installation place of the user interface device for manager.

Expression 6: The exercise menu management device according to Expression 5, in which the user executes the exercise menu in a place other than the installation place of the user interface device for manager.

Expression 7: The exercise menu management device according to Expression 6, in which, when the body state information cannot be measured in the installation place of the user interface device for manager, information transmitted from the user device is stored in the body state information storage part as the body state information.

Expression 8: The exercise menu management device according to Expression 6, in which, when the body state information cannot be measured in the installation place of the user interface device for manager, the body state information of the user is estimated from the latest body state information of the user and the execution status of the exercise menu, and is stored in the body state information storage part.

### REFERENCE SIGNS LIST

1: Exercise menu management device, 2, 2A: User device, 3, 3A, 3B: Exercise device, 11: Exercise menu creation part, 13: User management part, 14: Event management part, 15: Walking evaluation part, 12: Storage part, 121: User basic information, 122: Body state information, 123: Walking importance degree information, 124: Exercise information, 125: Exercise moving image management information 125, 126: Exercise execution record, 127: Walking record, 128: Walking evaluation standard, EM: Exercise menu, EMS: Exercise menu management system

1. An exercise menu management device that provides a user with an exercise menu related to improvement of a walking function, the exercise menu management device comprising:
a memory storing
body state information of a plurality of predetermined parts related to the walking function among parts of a body of the user,
walking importance degree information indicating a degree of importance related to the walking function for each of the plurality of predetermined parts, and
exercise information indicating a relationship between a plurality of exercises contributing to the improvement of the walking function and the plurality of predetermined parts; and
a processor programmed to
select one or more of the plurality of predetermined parts based on the body state information and the walking importance degree information,
create the exercise menu by selecting from the plurality of exercises a predetermined exercise related to the selected one or more predetermined parts, and
output the exercise menu that has been created.

2. The exercise menu management device according to claim 1, wherein the processor outputs the exercise menu to a user device used by the user.

3. The exercise menu management device according to claim 2, wherein
the exercise information further includes a degree of difficulty at the time of executing the plurality of exercises, and
the processor selects the predetermined exercise based on the degree of difficulty.

4. The exercise menu management device according to claim 2, wherein
the processor is further programmed to acquire and manage data related to walking of the user when the user participates in an event contributing to the improvement of the walking function.

5. An exercise menu management system comprising:
the exercise menu management device according to claim 2;
the user device; and
an exercise device used by the user, wherein
the user device includes a communication circuit that communicates with the exercise device, and
the exercise device includes a sensor that detects information of a time when the user performs an exercise, and a communication part that transmits the information detected by the sensor to the communication circuit.

6. The exercise menu management system according to claim 5, wherein
the exercise device further includes an information provision part, and the information provision part provides the user with information on the exercise.

7. The exercise menu management system according to claim 6, wherein
the exercise device includes a board part placed on a floor, a plurality of main body parts provided on the board part, and an attachment part provided in each of the main body parts so as to be expandable and attached to the body of the user,
each of the main body parts is configured to exert a force of pulling the attachment part separated from the respective main body part to the respective main body part,
the information provision part is provided in each of the main body parts, and
each of the information provision parts provides the user with the information related to the exercise by working together with each of the other information provision parts.

8. The exercise menu management device according to claim 2, wherein
the processor outputs the exercise menu to the user device, causes a moving image related to the exercise menu to be read from the memory, and causes the moving image to be distributed to the user device.

9. The exercise menu management device according to claim 8, wherein
the moving image is prepared according to a type of the user, and the processor causes the moving image according to the type of the user to be read from the memory and causes the moving image to be distributed to the user device.

10. An exercise menu management method of creating an exercise menu related to improvement of a walking function by an exercise menu management device and providing a user with the exercise menu, the method comprising:
storing, in a memory, body state information of a plurality of predetermined parts related to the walking function among parts of a body of the user;
storing, in the memory, walking importance degree information indicating a degree of importance related to the walking function for each of the plurality of predetermined parts;
storing, in the memory, exercise information indicating a relationship between a plurality of exercises contributing to the improvement of the walking function and the plurality of predetermined parts;
selecting, by a processor, one or more of the plurality of predetermined parts based on the body state information and the walking importance degree information;
creating, by the processor, the exercise menu by selecting from the plurality of exercises a predetermined exercise related to the selected one or more predetermined parts; and
outputting, by the processor, the exercise menu that has been created.

11. A non-transitory computer-readable medium storing thereon a program that causes a computer to execute:
storing, in a memory of the computer, body state information of a plurality of predetermined parts related to a walking function among parts of a body of the user;
storing, in the memory, walking importance degree information indicating a degree of importance related to the walking function for each of the plurality of predetermined parts;
storing, in the memory, exercise information indicating a relationship between a plurality of exercises contributing to improvement of the walking function and the plurality of predetermined parts;
selecting, by a processor of the computer, one or more of the plurality of predetermined parts based on the body state information and the walking importance degree information;
creating, by the processor, an exercise menu by selecting from the plurality of exercises a predetermined exercise related to the selected one or more predetermined parts; and
outputting, by the processor, the exercise menu that has been created.
